(19) United States
(12) Patent Application Publication
(10) Pub. No.: US 2026/0041906 A1
(43) Pub. Date: Feb. 12, 2026

(54) CONFIGURATIONS FOR ENDOVASCULAR THERAPY DEVICE

(71) Applicant: Covidien LP, Mansfield, MA (US)

(72) Inventors: Robert L. Olson, White Bear Lake, MN (US); Dale F. Seeley, Spring Park, MA (US)

(21) Appl. No.: 19/293,365

(22) Filed: Aug. 7, 2025

Related U.S. Application Data
(60) Provisional application No. 63/681,502, filed on Aug. 9, 2024.

(51) Int. Cl. A61N 1/05

(52) U.S. Cl. CPC A61N 1/05

(57) ABSTRACT

An endovascular medical device system includes an elongated body configured to be introduced in a blood vessel of a patient. The system includes an expandable structure at a distal portion of the elongated body. In some examples, the expandable structure includes a plurality of interconnected struts and extends from an expandable structure proximal end to an expandable structure distal end. The system further includes a plurality of electrodes carried by the expandable structure. The system includes further a plurality of conductor wires electrically connected to respective electrodes. In some examples, the plurality of conductor wires collectively form a multi-wire coil, the multi-wire coil extending along at least a portion of the elongated body and extending distally of the expandable structure proximal end. In some examples, a subset of conductor wires of the plurality of conductor wires branch out from the multi-wire coil to the respective electrodes.

### CROSS-REFERENCE TO RELATED APPLICATIONS

This application claims the benefit of and priority to U.S. Provisional Patent Application Ser. No. 63/681,502 filed Aug. 9, 2024, the entire disclosure of which is incorporated by reference herein.

### TECHNICAL FIELD

This disclosure relates to electrical stimulation therapy.

### BACKGROUND

Medical devices, such as electrical stimulation devices, may be used in different therapeutic applications, such as vagus nerve stimulation (VNS) and/or deep brain stimulation (DBS). A medical device may be used to deliver therapy to a patient to treat a variety of symptoms or patient conditions. In some therapy systems, an external or an implantable electrical stimulator delivers electrical stimulation therapy to a target tissue site within a patient with the aid of one or more electrodes and/or senses one or more patient parameters with the aid of the one or more electrodes.

### SUMMARY

This disclosure describes example endovascular medical devices and systems configured to endovascularly deliver electrical stimulation therapy to a patient (e.g., to one or more nerves or brain targets) and/or sense one or more patient parameters (e.g., nerve signals, brain signals, and/or other physiological parameters), and related methods. In particular, this disclosure describes configurations for structures of medical devices and systems that facilitate delivery of electrical stimulation therapy and/or sensing patient parameters from an endovascular location.

In the examples described herein, an endovascular therapy system includes one or more electrodes and/or other sensing elements that are carried by an expandable structure at a distal portion of an elongated body (e.g., a medical lead). The expandable structure (e.g., a stent, or stent-like structure) is configured to transform between a delivery (e.g., compressed or relatively low-profile) configuration and a deployed (e.g., expanded) configuration. One or more electrodes and/or sensing elements are mechanically coupled to, disposed on, or otherwise carried by the expandable structure. Each electrode and/or sensing element is electrically connected to a medical device via conductor wires. The medical device is configured to deliver therapy (e.g., electrical stimulation therapy) and/or received sensed signals via the electrodes by way of the conductor wires. The conductor wires can extend from the medical device, along the elongated body, and distal of the elongated body to electrically connect to each electrode or sensing element.

In some examples herein, the configurations of the interface and/or mechanical coupling (e.g., physical connection) between the elongated body and the expandable structure can reduce a mechanical load on the conductor wires and/or reduce fatigue of conductor wires, even during navigation of the elongated structure with conductor wires through vasculature of a patient, during transformation of the expandable structure between the delivery configuration and the deployed configuration, and/or while the elongated body and the expandable structure are positioned in the patient, e.g., for temporary or chronic therapy delivery or sensing. In some examples, a direct mechanical connection between the elongated body and the expandable structure can facilitate a reduction and/or elimination of mechanical forces and/or loads on the conductor wires (e.g., as compared to other medical device systems in which the elongated body and the expandable structure are not directly mechanically connected). Such reduced mechanical loads and/or loads, which may in turn lead to reduced fatigue of conductor wires, can enable use of thinner conductor wires, which may reduce the overall profile (e.g., cross-sectional profile) of the medical device system. A reduced overall profile of the medical device can facilitate relatively easier delivery and deployment of the medical device system proximate a target location, as well as reduce the risk of thrombus formation after the medical device system has been delivered and deployed at the target location. Further, the use of thinner conductor wires can enable the use of a greater number of conductor wires (e.g., a relatively greater number of conductor wires, as compared to other medical device systems), which may in turn enable the use of more electrodes and/or sensing elements. The use of more electrodes and/or sensing elements can facilitate a greater therapeutic effect of electrical stimulation therapy, more granular measurements from sensed signals, or improvement of other clinically relevant outcomes.

In some examples herein, the configurations of the interface and/or mechanical coupling (e.g., physical connection) between the elongated body and the expandable structure is configured to facilitate a mechanically robust connection between the elongated body (e.g., a medical lead) and the expandable structure while also being relatively low-profile and/or otherwise reducing disruption to blood flow proximate and/or through the expandable structure. For example, in some examples herein, the mechanical connection between the elongated body (e.g., a medical lead) and the expandable structure can enable some portions and/or components of the medical device system to reside closer to a vessel wall and/or apposed to a vessel wall (e.g., a blood vessel wall) after the device has been delivered and deployed at the target location. By having some portions closer to and/or opposed to a vessel wall when the device is in the deployed configuration proximate a target location, the system can reduce the impact to blood flow proximate and/or through the medical device system (e.g., as compared to other medical device system that tend to reside closer to a center of a vessel).

In some examples herein, configurations of conductor wires and/or other system elements associated with the conductor wires can facilitate an improved mechanical integrity and/or reduced fatigue of the conductor wires. For example, the conductor wires can include one or more multi-wire coil portions. Multi-wire coil portions can facilitate improved mechanical integrity and/or reduced fatigue of the conductor wires even during delivery and deployment of the medical device system within the vasculature of a patient.

In some examples herein, the medical device system additionally includes one or more conductor wire management tools (e.g., a conductor wire separator) configured to facilitate spacing, organization, and/or routing of conductor wires. Such conductor wire management tools can reduce or even eliminate mechanical load placed on conductor wires from crossing and/or tangling of wires (e.g., such as at particular transition locations of conductor wires, such as at locations where a multi-wire coil splits and each individual conductor wires extend out to electrically connect to individual electrodes). Additionally or alternatively, such conductor wire management tools can reduce the likelihood of unwanted shorting of conductor wires (e.g., due to degradation of electrically insulative material surrounding individual conductor wires that can cause unwanted electrical shorts between individual conductor wires). Additionally or alternatively, such conductor wire management tools can facilitate application of one or more coatings (e.g., antithrombogenic coatings) that is applied to one or more conductor wires (e.g., during a manufacturing and/or assembly process).

In some examples, an endovascular medical device system includes an elongated body configured to be introduced in a blood vessel of a patient; an expandable structure at a distal portion of the elongated body, the expandable structure including a plurality of interconnected struts and extending from an expandable structure proximal end to an expandable structure distal end; a plurality of electrodes carried by the expandable structure; and a plurality of conductor wires, each conductor wire of the plurality of conductor wires electrically connected to respective electrode of the plurality of electrodes, wherein the plurality of conductor wires collectively form a multi-wire coil, the multi-wire coil extending along at least a portion of the elongated body and extending distally of the expandable structure proximal end, and wherein at least a subset of conductor wires of the plurality of conductor wires branch out from the multi-wire coil to the respective electrodes of the plurality of electrodes to electrically connect to the respective electrodes.

In some examples, a method of using a medical device system includes introducing a medical device into vasculature of a patient, the medical device includes an elongated body configured to be introduced in a blood vessel of a patient; an expandable structure at a distal portion of the elongated body, the expandable structure including a plurality of interconnected struts and extending from an expandable structure proximal end to an expandable structure distal end; a plurality of electrodes carried by the expandable structure; and a plurality of conductor wires, each conductor wire of the plurality of conductor wires electrically connected to respective electrode of the plurality of electrodes, wherein the plurality of conductor wires collectively form a multi-wire coil, the multi-wire coil extending along at least a portion of the elongated body and extending distally of the expandable structure proximal end, and wherein at least a subset of conductor wires of the plurality of conductor wires branch out from the multi-wire coil to the respective electrodes of the plurality of electrodes to electrically connect to the respective electrodes; and advancing the medical device until the plurality of electrodes are at or near a target location in the vasculature of the patient.

In some examples, an endovascular medical device system includes an elongated body configured to be introduced in a blood vessel of a patient, the elongated body extending between an elongated body proximal end and an elongated body distal end; an expandable structure extending from an expandable structure proximal end to an expandable structure distal end, wherein the expandable structure is mechanically connected to the elongated body such that the elongated body distal end is distal to the expandable structure proximal end; a plurality of electrodes carried by the expandable structure; and a plurality of conductor wires, each conductor wire of the plurality of conductor wires electrically connected to respective electrodes of the plurality of electrodes, wherein the plurality of conductor wires collectively form a multi-wire coil, the multi-wire coil extending along at least a portion of the elongated body and extending distally of the expandable structure proximal end, wherein at least a subset of conductor wires of plurality of conductor wires branch out from the multi-wire coil to the respective electrodes of the plurality of electrodes to electrically connect to the respective electrodes, wherein the expandable structure is configured to transform from a relatively low-profile delivery configuration to a deployed configuration to position the plurality of electrodes to deliver electrical stimulation to tissue of the patient or sense a patient parameter from a location within the blood vessel, and wherein the plurality of conductor wires is configured to accommodate the transformation of the expandable structure from the relatively low-profile delivery configuration to the deployed configuration.

The examples described herein may be combined in any permutation or combination.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a conceptual diagram illustrating an example therapy system including an endovascular device configured to deliver electrical stimulation therapy to a target tissue site of a patient and/or sense a patient parameter from an endovascular location.

FIG. 2 is a functional block diagram illustrating components of an example medical device of the therapy system of FIG. 1.

FIG. 3A and FIG. 3B illustrate a distal portion of an example endovascular therapy system including an expandable structure and electrodes carried by the expandable structure.

FIG. 4 illustrates an example mechanical coupling between an expandable structure and a distal portion of an elongated body.

FIG. 5 illustrates an example mechanical coupling between an expandable structure and a distal portion of an elongated body.

FIG. 6 illustrates an example mechanical coupling between an expandable structure and a distal portion of an elongated body.

FIG. 7A and FIG. 7B illustrate an example radiopaque element that is circumferentially aligned with electrodes.

FIG. 8 illustrates an example multi-wire conductor coil splitting to form at least a first sub-coil and a second sub-coil.

FIG. 9A illustrates an example elongated body including a conductor wire management tool at a distal portion of the elongated body.

FIG. 9B and FIG. 9C illustrate the example conductor wire management tool from FIG. 9A.

FIG. 10A and FIG. 10B illustrate an example conductor wire management tool.

FIG. 11 illustrates a distal portion of an example endovascular therapy system including an expandable structure and electrodes carried by the expandable structure.

FIG. 12 illustrates a distal portion of an example endovascular therapy system including an expandable structure and electrodes carried by the expandable structure.

FIG. 13 is a flow diagram illustrating an example technique for introducing and advancing an endovascular device according to this disclosure.

Like reference characters denote like elements throughout the description and figures.

### DETAILED DESCRIPTION

This disclosure describes devices, systems, and methods relating to delivery of electrical stimulation therapy, such as vagus nerve stimulation (VNS), deep brain stimulation (DBS), and/or sensing one or more patient parameters (e.g., nerve activity from one more nerves, cardiac signals, muscle activation signals, brain signals and/or other physiological parameters, such as impedance, electroencephalogram (EEG), evoked potentials, local field potentials, etc.) from an endovascular location. Example endovascular locations that can be used for electrical stimulation therapy (e.g., VNS therapy) and/or sensing using the devices described herein include an internal jugular vein (IJV). Example endovascular locations that can be used to access the brain sites for electrical stimulation therapy (e.g., DBS) and/or sensing using the devices described herein include any suitable cranial blood vessel (also referred to herein as a cerebral blood vessel or neurovasculature, which can include a vein or an cranial artery), such as, but not limited to, the thalamostriate vein, the internal cerebral vein, the basal vein of Rosenthal, the inferior sagittal sinus, the superior sagittal sinus, or the anterior choroidal artery.

VNS has been proposed for use to manage one or more patient conditions, such as to control an inflammatory response in patients. Stimulating the vagus nerve may dampen the inflammatory response and associated cytokine response. In some examples, inflammatory cytokines are modulated up or down via stimulation. In addition, VNS may assist in stroke rehabilitation and limit ischemia reperfusion injury. After a myocardial infarct or stroke, reperfusion therapies (surgery or drugs) are given to restore blood flow. However, due to the restoration of blood, flow induced local damage occurs, including ischemia reperfusion injury. This injury may induce local accumulations of chemical mediators such as reactive oxygen species (ROS) production, inflammatory cytokines, bradykinin, etc., which can further affect inflammation. Such inflammatory compounds may trigger sensory signaling, which can lead to a reduced organ vagus activity and sympathetic overdrive. Vagus nerve stimulation may treat reperfusion damage as the inflammatory state may be lowered by increasing parasympathetic drive.

DBS has been proposed for use to manage one or more patient conditions. For example, DBS can be used to alleviate, and in some cases, eliminate symptoms associated with movement disorders, other neurodegenerative impairment, seizure disorders, psychiatric disorders (e.g., mood disorders), or the like. Movement disorders may be found in patients with Parkinson's disease, multiple sclerosis, and cerebral palsy, among other conditions, and can be associated with disease or trauma. DBS can be delivered to one or more target sites in a brain of a patient to help a patient with muscle control and minimize movement problems, such as rigidity, bradykinesia (i.e., slow physical movement), rhythmic hyperkinesia (e.g., tremor), nonrhythmic hyperkinesia (e.g., tics) or akinesia (i.e., a loss of physical movement).

In the case of seizure disorders, DBS can be delivered to one or more target sites in a brain of a patient to reduce the frequency or severity of seizures, or even help prevent the occurrence of seizures. In the case of psychiatric disorders, DBS can be delivered to help minimize or even eliminate symptoms associated with major depressive disorder (MDD), bipolar disorder, anxiety disorders, post-traumatic stress disorder, dysthymic disorder, or obsessive-compulsive disorder (OCD). DBS can also reduce the symptoms of Parkinson's disease, dystonia, or cerebellar outflow tremor.

While this disclosure is primarily directed to examples of VNS and/or sensing via applicable endovascular locations (e.g., the internal jugular vein), it should be understood that the devices, systems, and techniques may be adapted for DBS, other kinds of brain stimulation, peripheral nerve stimulation, or electrical stimulation and/or sensing of any nerve tissue that can be done via an endovascular location.

In the examples described herein, an endovascular therapy system includes one or more electrodes and/or other sensing elements that are carried by an expandable structure at a distal portion of an elongated body (e.g., a medical lead). The expandable structure (e.g., a stent, or stent-like structure) is configured to transform between a delivery (e.g., compressed or relatively low-profile) configuration and a deployed (e.g., expanded) configuration. One or more electrodes and/or sensing elements are mechanically coupled to, disposed on, or otherwise carried by the expandable structure. Each electrode and/or sensing element is electrically connected to a medical device via conductor wires. The medical device is configured to deliver therapy (e.g., electrical stimulation therapy) and/or received sensed signals via the electrodes by way of the conductor wires. The conductor wires may extend along, be a part of, incorporated into, and/or integrally formed as part of the elongated body (e.g., the medical lead). In some examples, conductor wires extend along (e.g., within) the elongated body (e.g., such as from a proximal portion of the elongated body to a location distal of the elongated body) to electrically connect each electrode or sensing element to the medical device.

In some examples herein, the configurations of the interface and/or mechanical coupling (e.g., physical connection) between the elongated body and the expandable structure can reduce a mechanical load on the conductor wires and/or reduce fatigue of conductor wires, even during navigation of the elongated structure with conductor wires through vasculature of a patient, during transformation of the expandable structure between the delivery configuration and the deployed configuration, and/or while the elongated body and the expandable structure are positioned in the patient, e.g., for temporary or chronic therapy delivery or sensing. In some examples, a direct mechanical connection between the elongated body and the expandable structure can facilitate a reduction and/or elimination of mechanical forces and/or loads on the conductor wires (e.g., as compared to other medical device systems in which the elongated body and the expandable structure are not directly mechanically connected). Such reduced mechanical loads and/or loads, which may in turn lead to reduced fatigue of conductor wires, can enable use of thinner conductor wires, which may reduce the overall profile (e.g., cross-sectional profile) of the medical device system. A reduced overall profile of the medical device can facilitate relatively easier delivery and deployment of the medical device system proximate a target location, as well as reduce the risk of thrombus formation after the medical device system has been delivered and deployed at the target location. Further, the use of thinner conductor wires can enable the use of a greater number of conductor wires (e.g., a relatively greater number of conductor wires, as compared to other medical device systems), which may in turn enable the use of more electrodes and/or sensing elements. The use of more electrodes and/or sensing elements can facilitate a greater therapeutic effect of electrical stimulation therapy, more granular measurements from sensed signals, or improvement of other clinically relevant outcomes.

In some examples herein, the configurations of the interface and/or mechanical coupling (e.g., physical connection) between the elongated body and the expandable structure is configured to facilitate a mechanically robust connection between the elongated body (e.g., a medical lead) and the expandable structure while also being relatively low-profile and/or otherwise reducing disruption to blood flow proximate and/or through the expandable structure. For example, in some examples herein, the mechanical connection between the elongated body (e.g., a medical lead) and the expandable structure can enable some portions and/or components of the medical device system to reside closer to a vessel wall and/or apposed to a vessel wall (e.g., a blood vessel wall) after the device has been delivered and deployed at the target location. By having some portions closer to and/or opposed to a vessel wall when the device is in the deployed configuration proximate a target location, the system can reduce the impact to blood flow proximate and/or through the medical device system (e.g., as compared to other medical device system that tend to reside closer to a center of a vessel).

In some examples herein, configurations of conductor wires and/or other system elements associated with the conductor wires can facilitate an improved mechanical integrity and/or reduced fatigue of the conductor wires. For example, the conductor wires can include one or more multi-wire coil portions. Multi-wire coil portions can facilitate improved mechanical integrity and/or reduced fatigue of the conductor wires even during delivery and deployment of the medical device system within the vasculature of a patient.

In some examples herein, the medical device system additionally includes one or more conductor wire management tools (e.g., a conductor wire separator) configured to facilitate spacing, organization, and/or routing of conductor wires. Such conductor wire management tools can reduce or even eliminate mechanical load placed on conductor wires from crossing and/or tangling of wires (e.g., such as at particular transition locations of conductor wires, such as at locations where a multi-wire coil splits and each individual conductor wires extend out to electrically connect to individual electrodes). Additionally or alternatively, such conductor wire management tools can reduce the likelihood of unwanted shorting of conductor wires (e.g., due to degradation of electrically insulative material surrounding individual conductor wires that can cause unwanted electrical shorts between individual conductor wires). Additionally or alternatively, such conductor wire management tools can facilitate application of one or more coatings (e.g., antithrombogenic coatings) that is applied to one or more conductor wires (e.g., during a manufacturing and/or assembly process).

In some examples, a medical device is configured to generate electrical stimulation and/or sense a patient parameter via the electrodes of the endovascular device. The electrodes may be carried by or otherwise disposed on an expandable structure, which may be configured to orient the electrodes and/or anchor the electrodes at a particular location in the vasculature of the patient.

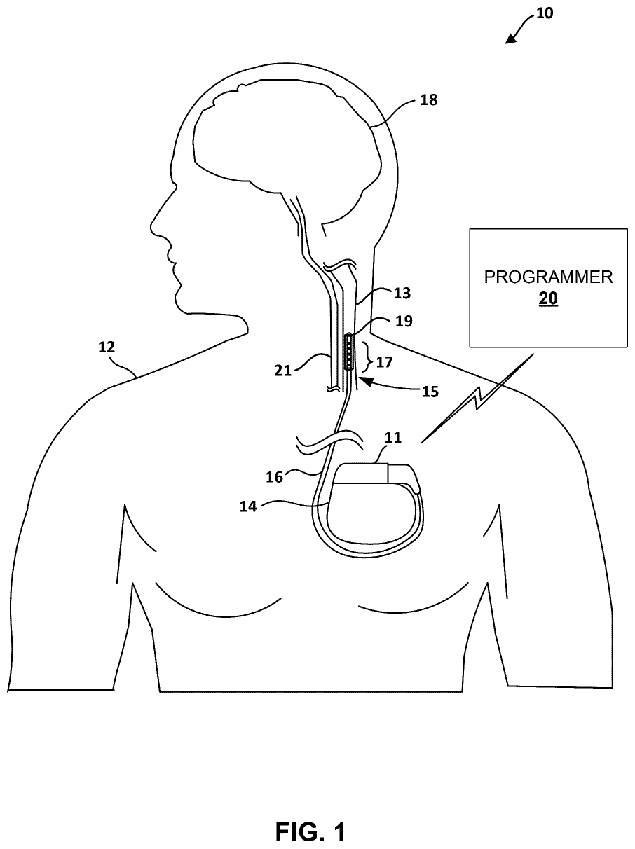

FIG. 1 is a conceptual diagram illustrating an example therapy system 10 configured to deliver electrical stimulation therapy to a target tissue site of a patient 12 or sense a patient parameter from an endovascular location. Patient 12 ordinarily will be a human patient. In some cases, however, therapy system 10 is applied to other mammalian or non-mammalian non-human patients. Therapy system 10 includes a medical device 14 and an endovascular device 16. In the example shown in FIG. 1, medical device 14 is configured to deliver electrical stimulation therapy (e.g., VNS) to a vagus nerve 21 of patient 12 and/or sense bioelectric signals via electrodes 17. However, in other examples, therapy system 10 and/or medical device 14 is configured to deliver electrical stimulation therapy (e.g., DBS) to brain 18 of patient 12 and/or sense bioelectrical brain signals in brain 18 via electrodes 17.

In the example of FIG. 1, endovascular device 16 is positioned in a jugular vein 13 of patient 12 such that one or more electrodes 17 are located proximate to a target tissue site. In particular, electrodes 17 are positioned to deliver electrical stimulation therapy to and/or sense signals from nerves surrounding jugular vein 13, including (but not limited to) vagus nerve 21. Endovascular device 16 includes an expandable structure 19 at a distal portion 15 of endovascular device 16 which may help hold electrodes 17 in apposition with a vessel wall (e.g., of jugular vein 13). In some examples, as discussed in relation to later examples, expandable structure 19 is mechanically coupled (e.g., directly mechanically coupled) to a portion of endovascular device 16 via a suitable mechanical connection (e.g., welding, crimped connection, or the like). Medical device 14 can provide electrical stimulation to one or more regions surrounding jugular vein 13 in order to manage a condition of patient 12, such as to mitigate the severity or duration of the patient condition.

Endovascular device 16 includes any suitable medical device configured to deliver electrical stimulation signals to tissue proximate electrodes 17. For example, endovascular device 16 can be a medical lead, a catheter, a guidewire, or another elongated body carrying electrodes 17 and configured to be electrically coupled to medical device 14 via an electrically conductive pathway (e.g., via one or more conductor wires) that runs between medical device 14 and electrodes 17. Endovascular device 16 has any suitable length that enables connection to medical device 14 either directly or indirectly, e.g., a length of 150 centimeters (cm) to 250 cm, such as 200 cm. Further, endovascular device 16 has a suitable length (e.g., as measured along a longitudinal axis of endovascular device 16) for accessing a target tissue site within the patient from a vascular access point. In examples in which endovascular device 16 accesses the jugular vein 13 and/or vasculature in a brain 18 of patient 12 from a femoral artery access point at the groin of the patient, endovascular device 16 has a length of about 100 cm to about 200 cm, although other lengths may be used. However, other access points may be used to introduce endovascular device 16 into vasculature of a patient, such as, but not limited to, a radial artery.

As used herein, “about” may indicate the exact value or nearly the exact value to the extent permitted by manufacturing tolerances. “About” can also refer to a certain percentage of the recited value (e.g., within about 1%, 5%, or 10%).

Endovascular device 16 is configured to be introduced in the vasculature of patient 12, such as to access jugular vein 13 and/or relatively more distal locations in a patient, such as the middle cerebral artery (MCA) in a brain of a patient. Endovascular device 16 may include an elongated body that is structurally configured to be relatively flexible, pushable, and relatively kink- and buckle-resistant, so that it may resist buckling when a pushing force is applied to a relatively proximal portion to advance endovascular device 16 distally through vasculature, and so that it may resist kinking when traversing around a tight turn in the vasculature. Kinking and/or buckling of may hinder a clinician's efforts to push the elongated body distally, e.g., past a turn. In some examples, endovascular device 16 includes one or more radiopaque components (e.g., platinum bands) proximate electrodes 17 and/or expandable structure 19.

Instead of or in addition to the elongated body of endovascular device 16 being configured for intravascular navigation to a cerebral blood vessel to deliver electrical stimulation therapy or sense a patient parameter, endovascular device 16 can be navigated through vasculature (e.g., to jugular vein 13, brain 18, or other target tissue sites) with the aid of a guide member. The guide member can include an outer catheter, an inner catheter, a guide extension catheter, a guidewire, or the like or combination thereof.

In some examples, more than one of endovascular device 16 is introduced into, positioned in, and/or implanted within patient 12 to provide stimulation to and/or sense multiple anatomical regions, including one or more of both the left and right jugular veins, as well as in locations of brain 18. For example, two or more of endovascular device 16, which may be paired with one or more of medical device 14, may be configured of bilateral stimulation and/or sensing (e.g., of the left jugular vein and a right jugular vein). Endovascular device 16, including electrodes 17 and/or expandable structure 19, can be positioned in and/or implanted within a blood vessel for chronic therapy delivery and/or chronic sensing (e.g., on the order of months or even years) or for more temporary therapy delivery and/or sensing (e.g., on the order of days, such as less than a month or less than 6 months). Temporary therapy delivery may include one or more trial periods, such as to determine, evaluate, or confirm an efficacy of stimulation and/or sensing, and/or to select electrical stimulation parameters for chronic therapy delivery.

The electrical stimulation therapy described herein (e.g., VNS, DBS, or the like) may be used to treat various patient conditions, such as, a variety of illnesses including, but not limited to: reperfusion damage, cardiac ischemia, brain ischemia, stroke, traumatic brain injury, surgical or non-surgical acute kidney injury, inability of the intestine (bowel) to contract normally and move waste out of the body, postoperative ileus, postoperative cognitive decline or postoperative delirium, asthma, sepsis, bleeding control, myocardial infarction reduction, dysmotility, obesity, movement disorders, other neurodegenerative impairment, seizure disorders, psychiatric disorders (e.g., mood disorders). Treating any of these diseases may improve patient outcomes by shortening length of hospital stays and reducing medical costs.

The vasculature into which endovascular device 16 may be inserted and/or guided includes, but is not limited to, veins or arteries. For example, endovascular device 16 can be navigated from a vasculature access site (e.g., in the femoral artery, the radial artery, or another suitable access site) to one or more of a jugular vein (e.g., internal jugular vein and/or external jugular vein), a carotid artery (e.g., internal carotid artery, external carotid artery, and/or common carotid artery), as well as brain targets including the thalamostriate vein, the internal cerebral vein, the basal vein of Rosenthal, the inferior/superior sagittal sinus, the anterior choroidal artery, or any related combinations thereof.

A clinician can also select a particular blood vessel to position electrodes 17 within, such as to avoid certain regions to minimize or even eliminate adverse effects. For example, electrodes 17 can be oriented or positioned relative to vagus nerve 21 to avoid inadvertently providing electrical stimulation to anatomical regions (e.g., undesired anatomical regions) near the targeted anatomical region.

In some examples, endovascular device 16 is configured to be delivered to one or more target sites in vasculature of patient 12. Thus, rather than introducing endovascular device 16 into tissue in close proximity with vagus nerve 21 through an incision in the neck or chest area of patient 12, endovascular device 16 is configured to be navigated proximate to a target electrical stimulation site via vasculature of patient 12. The endovascular delivery of endovascular device 16 to target sites can help minimize the invasiveness of therapy system 10.

In some examples, one or more electrodes 17 are positioned on (e.g., mechanically coupled to, defined by, or otherwise carried by) expandable structure 19 of endovascular device 16, which is configured to expand radially outwards from a relatively low-profile (e.g., radially compressed) delivery configuration to a deployed configuration. This may enable electrodes 17 to be held in apposition with a blood vessel wall, promote tissue ingrowth around electrodes 17 along the vessel wall (while still leaving a patent lumen to enable blood flow through the blood vessel, through expandable structure 19, despite implantation of endovascular device 16), which can reduce the overall power needed to deliver efficacious electrical stimulation therapy to a target tissue site, and help secure electrodes 17 in place in the blood vessel for chronic therapy delivery.

Medical device 14 can be an external medical device or an implantable medical device that includes electrical stimulation circuitry configured to generate and deliver electrical stimulation therapy to patient 12 and/or sensing circuitry configured to sense a patient parameter (e.g., a physiological signal) via one or more electrodes 17 of endovascular device 16. In the example shown in FIG. 1, endovascular device 16 is directly or indirectly mechanically and electrically coupled to medical device 14 via a header 11 of medical device 14, which defines a plurality of electrical contacts in one or more feedthrough portions (e.g., that are configured to electrically couple electrodes 17 to electrical stimulation generation circuitry and/or sensing circuitry within medical device 14).

In some examples, therapy system 10 includes one or more conductor wires (not shown in FIG. 1) extending between medical device 14 and electrodes 17, the one or more conductor configured to carry electrical signals between medical device and electrodes 17 or vice versa. The conductor wires may extend along, be a part of, incorporated into, and/or integrally formed as part of endovascular device 16. In some examples, header 11 includes multiple feedthrough portions, which may be respectively configured for receiving one of multiple portions of endovascular device 16. Header 11 may also be referred to as a connector block or connector of medical device 14. Endovascular device 16 may be mechanically coupled and/or electrically coupled to header 11 with the aid of a lead extension. However, in some examples, a lead extension is not used between header 11 and endovascular device 16, and endovascular device is directly mechanically and/or electrically connected to medical device 14 via header 11.

In some examples, medical device 14 is configured to be positioned in (e.g., implanted in) patient 12 in any suitable location, such as a location in a pectoral region. In other examples, medical device 14 is configured to be external to patient 12. Endovascular device 16 may be, for example, implanted within a vein (e.g., jugular vein 13) and one or more proximal wires/leads can remain within the venous system until they exit the venous system, such as through the subclavian vein in the chest or the internal jugular vein in the neck for implant in the pectoral region. In yet other examples, some or all of medical device 14 is configured to be implanted in the vasculature, e.g., as part of endovascular device 16.

As shown in FIG. 1, system 10 may also include a programmer 20, which may be a handheld device, portable computer, or workstation that provides a user interface to a user, for example a clinician or other user, such as a patient. The user may interact with the user interface to program electrical stimulation parameters for medical device 14.

With the aid of programmer 20 or another computing device, a clinician may select values for therapy parameters for controlling therapy delivery by therapy system 10. The values for the therapy parameters may be organized into a group of parameter values referred to as a “therapy program” or “therapy parameter set.” “Therapy program” and “therapy parameter set” are used interchangeably herein. In the case of electrical stimulation, the therapy parameters may include a combination of activated electrodes (also referred to herein as an electrode combination), a power, and an amplitude, which may be a current or voltage amplitude, and, if medical device 14 delivers electrical pulses, a pulse width, and a pulse rate for stimulation signals to be delivered to the patient. Other example therapy parameters include a slew rate, duty cycle, and phase of the electrical stimulation signal.

An electrode combination may include a selected subset of one or more electrodes 17 located on one or more of endovascular devices 16 mechanically coupled and/or electrically coupled to medical device 14. The electrode combination may also refer to the polarities of the electrodes in the selected subset. By selecting particular electrode combinations, a user may target particular tissue sites (e.g., anatomic structures) within patient 12. In addition, by selecting values for slew rate, duty cycle, phase amplitude, pulse width, and/or pulse rate, the user can attempt to generate an efficacious therapy for patient 12 that is delivered via the selected electrode subset.

Whether programmer 20 is configured for clinician or patient use, programmer 20 may be configured to communicate with medical device 14 or any other computing device via wireless or a wired communication. Programmer 20, for example, may communicate via wireless communication with medical device 14 using radio frequency (RF) telemetry techniques. Programmer 20 may also communicate with another programmer or computing device via a wired or wireless connection using any of a variety of local wireless communication techniques, such as RF communication according to the 802.11 or Bluetooth specification sets, infrared communication according to the Infrared Data Association (IRDA) specification set, or other standard or proprietary telemetry protocols. Programmer 20 may also communicate with another programming or computing device via a wired or wireless communication technique.

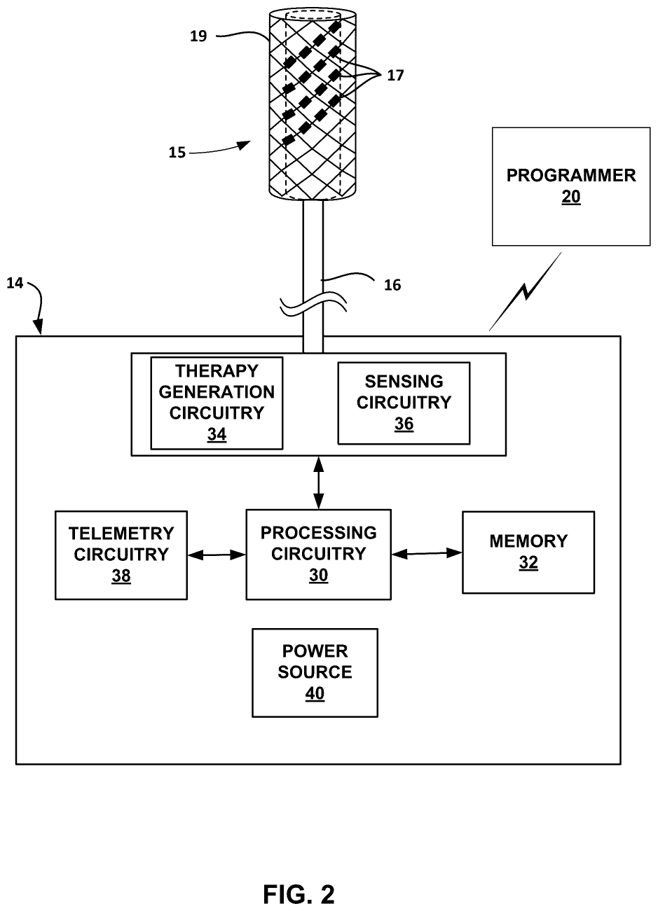

In some examples, in addition to or instead of delivering electrical stimulation to a target location (e.g., vagus nerve 21), medical device 14 or another device is configured to sense one or more patient parameters, such as bioelectric signals, either using electrodes 17 or other types of sensors that are carried by endovascular device 16. Bioelectric signals (also referred to herein as bioelectrical signals) can be sensed, and indications of sensed signals can be used by clinicians to make clinically relevant decision. In other examples, sense bioelectric signals are used as part of continuous feedback system in which medical device 14 adjusts one or more therapy parameter values based on sensed bioelectrical signals. Example bioelectric signals are described in further detail below with reference to FIG. 2.

In some examples, medical device 14 is configured to generate and deliver a suitable electrical stimulation signal, which can be a continuous time signal (e.g., a sinusoidal waveform or the like) or a plurality of pulses. In some examples, the electrical stimulation waveform generated by medical device 14 and delivered by one or more of electrodes 17 is a charge balanced, biphasic waveform. In some examples, such an electrical stimulation waveform consists of periodic pulses or otherwise include periodic pulses, or can include a continuous time waveform.

As noted above, in some examples, one or more electrodes 17 are positioned on expandable structure 19. In some examples, one or more sensors that are different from electrodes 17 are positioned on the same expandable structure (e.g., expandable structure 19) as one or more electrodes 17 or on a different expandable structure (e.g., a structure similar to or different from expandable structure 19) of endovascular device 16. Expandable structure 19 can have any suitable configuration that enables endovascular device 16 to assume a relatively low-profile configuration (also referred to herein as a “delivery” or “compressed” configuration in some examples) to facilitate delivery through vasculature to a target tissue site and expand radially outwards (relative to a central longitudinal axis of endovascular device 16) to position the one or more electrodes 17 closer to target tissue.

In some examples, expandable structure 19 is configured to expand radially outwards with sufficient force and to a cross-sectional dimension (e.g., a diameter) sufficient to position the one or more electrodes 17 in apposition with a blood vessel wall. Positioning one or more electrodes 17 in apposition with a blood vessel wall may help promote tissue ingrowth around electrodes 17, which can reduce the impedance and the overall power needed to deliver efficacious electrical stimulation therapy to a target tissue site, and help secure electrodes 17 in place in the blood vessel for chronic (e.g., on the order of months or even years) therapy delivery. Fixing endovascular device 16 in place within the blood vessel via the tissue ingrowth or, in some examples, using another fixation structures/anchoring mechanisms, such as tines, coils, barbs, or the like, can also help reduce the possibility of thrombosis.

Expandable structure 19 can be configured to expand radially outwards using any suitable technique and configuration. In some examples, expandable structure 19 includes a shape memory (e.g., nitinol) material that enables expandable structure 19 to assume a predetermined shape in the absence of a force (e.g., a compressive or tensile force) holding expandable structure 19 in a relatively low-profile delivery configuration. For example, expandable structure 19 can be configured to expand (e.g., self-expand) radially outwards upon deployment from an outer sheath (e.g., an outer catheter), or upon the proximal withdrawal of a straightening element (e.g., a guidewire or a mandrel) positioned in an inner lumen of the endovascular device 16. In some examples, expandable structure 19 is configured to expand radially outwards in response to proximal movement of a pull member attached to a distal portion of the endovascular device 16, in response to a distal movement of an elongated control member attached to the expandable structure, or with the aid of a balloon or the like.

Expandable structure 19 can have any suitable configuration in its deployed (e.g., expanded) configuration. In some examples herein, expandable structure 19 includes a plurality of interconnected struts to form a structure configured to expand radially outward (e.g., from a central longitudinal axis of expandable structure 19). For example, expandable structure 19 can include a tubular member, a basket, include one or more splines or arms configured to expand radially outwards, define one or more loops, define a helical or spiral element, or the like or combinations thereof, when in the deployed configuration. One or more expandable structures 19 may be disposed at various positions along endovascular device 16 (e.g., at one or more longitudinal positions along endovascular device 16). Expandable structure 19 can be formed from a plurality of structural elements (e.g., braided or mechanically coupled together) or can be a unitary structure (e.g., a laser cut nitinol tube). In some examples, expandable structure 19 is referred to herein as having a stent-like structure.

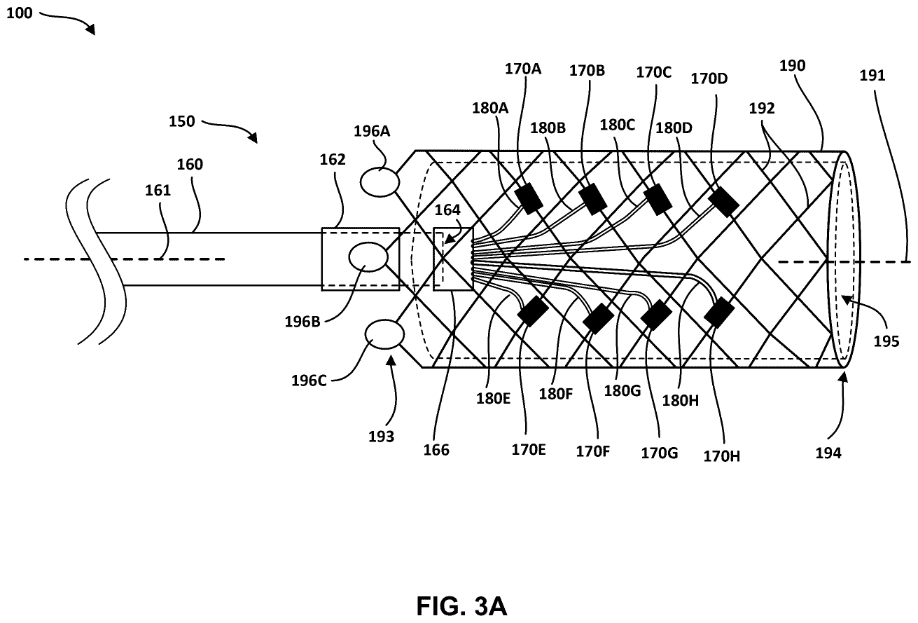

As discussed in relation to later examples (e.g., FIG. 3A and FIG. 3B), expandable structure 19 can be mechanically coupled to a portion of endovascular device 16 (e.g., distal portion 15 of endovascular device 16). In some examples, expandable structure 19 is mechanically coupled to endovascular device 16 via a welded connection, a crimped connection, a bonded connection (e.g., via an adhesive and/or another suitable bonding agent), or another suitable mechanical connection. In some examples herein, the mechanical connection between expandable structure 19 endovascular device 16 can facilitate a reduced mechanical load on and/or reduced mechanical fatigue of various components of therapy system 10. For example, the mechanical connection between expandable structure 19 endovascular device 16 can facilitate a reduced mechanical load on and/or reduced mechanical fatigue of one or more conductor wires (not shown in FIG. 1) extending between electrodes 17 and medical device 14.

In addition to, or instead of, chronic therapy delivery and/or chronic sensing, example devices, systems, and methods described herein can be used for more temporary applications. In some examples, a first endovascular device (e.g., configured like endovascular device 16 or having another configuration) is configured to be operated in an acute (e.g., temporary) trial mode for a trial period to determine, evaluate, or confirm an efficacy of stimulation and/or sensing. For example, endovascular device 16 (as well as electrodes 17, medical device 14, processing circuitry, etc.) may be configured to operate in the trial mode to determine the efficacy of one or more stimulation parameter values and/or one or more sensing parameters. After the acute trial period, the first endovascular device may be removed, and a second endovascular device (e.g., configured like endovascular device 16 or having another configuration) configured to operate in a chronic mode may be implanted for a chronic period for chronic (e.g., long term, or permanent) stimulation therapy or sensing. In some examples, a first endovascular device (e.g., for use in the acute trial mode) is configured to be implanted and subsequently removed after the trial period.

A trial period has a shorter intended duration as compared to a chronic period, though the ultimate length of the chronic period may be less than an intended duration due to one or more factors, such as a patient response that requires shortening the chronic period relative to the intended duration of the chronic period. In some examples, the trial period includes a trial period length on the order of minutes (e.g., 1 minute, 2 minutes, 3 minutes, 5 minutes, 30 minutes, 45 minutes, etc.), on the order of hours (e.g., 1 hour, 2 hours, 5 hours, 12 hours, etc.), on the order of days (e.g., 1 day, 2 days, 3 days, etc.), on the order of weeks (e.g., 1 week, 2 weeks, 3 weeks, etc.) on the order of months (e.g., 1 month, 2 months, 3 months, etc.), or longer. In some examples, one or more of endovascular devices may be used for multiple trial periods (e.g., successive trial periods) for determining an efficacy of one or more stimulation parameters and/or one or more sensing parameters.

Therapy system 10 may have any suitable configuration for delivering electrical stimulation to a target tissue site in patient 12 or sensing a patient parameter from an endovascular location (e.g., jugular vein 13). In some examples, therapy system 10 includes a first subset of electrodes of electrodes 17 configured for delivering electrical stimulation therapy and a second subset of electrodes of electrodes 17 configured to for sensing one or more patient parameters. In some examples, some or all electrodes of electrodes 17 are configured for both electrical stimulation therapy and for sensing one or more patient parameters. Therapy system 10 can include any suitable number of electrodes 17 and/or combination of different kinds of electrodes. In some examples, electrodes 17 include electrodes formed via one or more manufacturing processes. For example, electrodes 17 can include a first electrode type (e.g., an electrode configured for delivery of electrical stimulation therapy), a second electrode type (e.g., an electrode configured to sensing a signal), or any suitable combination thereof.

FIG. 2 is a functional block diagram illustrating components of an example medical device 14, which is configured to generate and deliver electrical stimulation therapy to patient 12 and, in some examples, sense one or more patient parameters, such as bioelectrical signals or other physiological parameter of patient 12. Medical device 14 includes processing circuitry 30, memory 32, therapy generation circuitry 34, sensing circuitry 36, telemetry circuitry 38, and power source 40.

Therapy generation circuitry 34 includes any suitable configuration (e.g., hardware) configured to generate and deliver electrical stimulation signals to target tissue (e.g., vagus nerve 21) in patient 12. Processing circuitry 30 is configured to control therapy generation circuitry 34 to generate and deliver electrical stimulation therapy via electrodes 17 of endovascular device 16. The therapy parameter values may be selected based on the patient condition being addressed, as well as the target tissue site in patient 12 for the electrical stimulation therapy. The electrical stimulation therapy can be provided via stimulation signals of any suitable form, such of stimulation pulses or continuous-time signals (e.g., sine waves).

Sensing circuitry 36 is configured to sense a physiological parameter of a patient. Sensing circuitry 36 may include any sensing hardware configured to sense a physiological parameter of a patient, such as, but not limited to, one or more electrodes, optical receivers, pressure sensors, or the like. The one or more sensing electrodes can be the same or different from electrodes 17 configured to deliver electrical stimulation therapy. In some examples, processing circuitry 30 stores the sensed physiological parameters in memory 32 or transmits the sensed parameters to another device via telemetry circuitry 38. In addition, in some examples, processing circuitry 30 can use the sensed physiological signals to control therapy delivery by therapy generation circuitry 34, e.g., the timing of the therapy delivery or one or more characteristics (e.g., parameters values) of the electrical simulation signal generated by therapy generation circuitry 34.

In some examples, sensing circuitry 36 is configured to sense a bioelectrical signal, which otherwise may be referred to as a patient parameter, via one or more electrodes 17 (e.g., all or a subset of electrodes 17). Thus, electrodes 17 can be configured to receive or transmit energy (e.g., current). In some examples, such as those in which electrodes 17 are placed proximate vagus nerve 21 (FIG. 1), example bioelectrical signals include muscle activation signals (e.g., laryngeal muscle activation), electrocardiogram (ECG), intracardiac electrogram (EGM), electromyogram (EMG). In other examples, such as those in which electrodes 17 are placed in or otherwise proximate brain 18, example bioelectrical signals include brain signals such as an EEG signal, an electrocorticogram (ECoG) signal, a signal generated from measured field potentials within one or more regions of brain 18, action potentials from single cells within brain 18 (referred to as “spikes”), or evoked potentials. Determining action potentials of single cells within brain 18 may require resolution of bioelectrical signals to the cellular level and provides fidelity for fine movements, i.e., a bioelectrical signal indicative of fine movements (e.g., slight movement of a finger).

In examples in which endovascular device 16 is configured to sense an evoked potential, endovascular device 16 may also be configured to generate a stimulus (e.g., via therapy generation circuitry 34, alone or in combination with processing circuitry 30) to elicit the evoked potential. For example, endovascular device 16 can generate and deliver electrical stimulation to tissue in brain 18 and sense an evoked compound action potential (ECAP). An ECAP is synchronous firing of a population of neurons which occurs in response to the application of a stimulus including, in some cases, an electrical stimulus by endovascular device 16. The ECAP may be detectable as being a separate event from the stimulus itself, and the ECAP may reveal characteristics of the effect of the stimulus on the tissue.

In some examples, sensing circuitry 36 and/or processing circuitry 30 includes signal processing circuitry configured to perform any suitable analog conditioning of the sensed physiological signals. For example, sensing circuitry 36 may communicate to processing circuitry 30 an unaltered (e.g., raw) signal. Processing circuitry 30 may be configured to modify a raw signal to a usable signal by, for example, filtering (e.g., low pass, high pass, band pass, notch, or any other suitable filtering), amplifying, performing an operation on the received signal (e.g., taking a derivative, averaging), performing any other suitable signal conditioning (e.g., converting a current signal to a voltage signal), or any combination thereof. In some examples, the conditioned analog signals are processed by an analog-to-digital converter of processing circuitry 30 or other component to convert the conditioned analog signals into digital signals. In some examples, processing circuitry 30 operates on the analog or digital form of the signals to separate out different components of the signals. In some examples, sensing circuitry 36 and/or processing circuitry 30 performs any suitable digital conditioning of the converted digital signals, such as low pass, high pass, band pass, notch, averaging, or any other suitable filtering, amplifying, performing an operation on the signal, performing any other suitable digital conditioning, or any combination thereof. Additionally or alternatively, sensing circuitry 36 may include signal processing circuitry to modify one or more raw signals and communicate to processing circuitry 30 one or more modified signals.

In some examples, processing circuitry 30, alone or in combination with therapy generation circuitry 34 and/or sensing circuitry 36, is configured to operate medical device 14 (including electrodes 17, endovascular device 16, etc.) in a trial mode for a trial period to determine an efficacy of electrical stimulation or sensing. As described above, a trial mode can include a trial period of stimulation and/or sensing to determine, evaluate, or confirm an efficacy of stimulation and/or sensing. In some examples, processing circuitry 30, alone or in combination with therapy generation circuitry 34 and/or sensing circuitry 36, is configured to deliver electrical stimulation therapy and/or sense a patient parameter during the trial period. In some examples, processing circuitry 30 is configured to determine, evaluate, or confirm an efficacy of stimulation and/or sensing. For example, processing circuitry 30 may determine one or more therapy parameters for chronic stimulation and/or sensing based on the trial period.

Although shown as part of medical device 14 in FIG. 2, in other examples, sensing circuitry 36 is part of a device separate from medical device 14. For example, sensing circuitry 36 can be part of an implantable sensing device implanted in patient 12.

Processing circuitry 30, as well as other processors, processing circuitry, controllers, control circuitry, and the like, described herein, may include any combination of integrated circuitry, discrete logic circuitry, analog circuitry, such as one or more microprocessors, digital signal processors (DSPs), application specific integrated circuits (ASICs), or field-programmable gate arrays (FPGAs). In some examples, processing circuitry 30 includes multiple components, such as any combination of one or more microprocessors, one or more DSPs, one or more ASICs, or one or more FPGAs, as well as other discrete or integrated logic circuitry, and/or analog circuitry.

Memory 32 is configured to store program instructions, such as software, which may include one or more program modules, which are executable by processing circuitry 30. When executed by processing circuitry 30, such program instructions may cause processing circuitry 30 to provide the functionality ascribed to processing circuitry 30 herein. The program instructions may be embodied in software and/or firmware. Memory 32 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media.

Processing circuitry 30 is configured to control telemetry circuitry 38 to send and receive information. Telemetry circuitry 38, as well as telemetry modules in other devices described herein, such as programmer 20 (FIG. 1), may accomplish communication by any suitable communication techniques, such as RF communication techniques. In addition, telemetry circuitry 38 may communicate with external medical device programmer 20 via proximal inductive interaction of medical device 14 with programmer 20. Accordingly, telemetry circuitry 38 may send information to external programmer 20 on a continuous basis, at periodic intervals, or upon request from medical device 14 or programmer 20.

Power source 40 is configured to deliver operating power to various components of medical device 14. Power source 40 may include a small rechargeable or non-rechargeable battery and a power generation circuit to produce the operating power. Recharging may be accomplished through proximal inductive interaction between an external charger and an inductive charging coil within medical device 14. In some examples, power requirements may be small enough to allow medical device 14 to utilize patient motion and implement a kinetic energy-scavenging device to trickle charge a rechargeable battery. In other examples, traditional batteries may be used for a limited period of time.

In some examples, endovascular device 16 is configured to be a standalone electrical stimulation device and can include one or more elements of medical device 14 shown in FIG. 2.

FIG. 3A and FIG. 3B illustrate an example endovascular therapy system 100, which is an example of therapy system 10 of FIG. 1. FIG. 3A illustrates a side view of endovascular therapy system 100, and FIG. 3B illustrates the side view of system 100 with some components at least partially transparent (e.g., removed) for illustration of the other components not fully shown in the example of FIG. 3A. Endovascular therapy system 100 includes a medical lead 160 and an expandable structure 190 at a distal portion 150 of medical lead 160. As shown, endovascular therapy system 100 includes electrode 170A, electrode 170B, electrode 170C, electrode 170D, electrode 170E, electrode 170F, electrode 170G, and electrode 170H, collectively referred to herein as electrodes 170. Medical lead 160, expandable structure 190, distal portion 150, and electrodes 170 are examples of endovascular device 16, expandable structure 19, distal portion 15, and electrodes 17 as shown and described in connection with FIG. 1, respectively.

Medical lead 160 can have any suitable configuration, and may be configured according to the description of endovascular device 16 of FIG. 1. In some examples, medical lead 160 includes an elongated body (e.g., a tubular body defining a lumen) extending between an elongated body proximal end (not show in the examples of FIG. 3A and FIG. 3B) and an elongated body distal end 164. In some examples, the elongated body of medical lead 160 defines an elongated body central longitudinal axis 161 extending along the elongated body of medical lead 160. Elongated body central longitudinal axis 161 may be a central longitudinal axis of medical lead 160.

In some examples, medical lead 160 includes a suitable biocompatible polymer material. For example, medical lead 160 can include a thermoplastic material, such as Polycarbonate Urethane (PCU). In some examples, medical lead can additionally or alternatively include one or more of Polyurethane (PUR or PU), Polyethylene (PE), Polypropylene (PP), Polyetheretherketone (PEEK), Polyphenylsulfone (PPSU or PPSF), Polypropylene (PP), Nylon, Polyester, Polyethlene Terephthalate (PET), Polymethyl Methacrylate (PMMA), Polysulfone (PSU), and/or another suitable material.

In some examples, medical lead 160 is configured to be at least partially introduced into, positioned in, and/or implanted within vasculature (e.g., blood vessel) of patient 12. In some examples, medical lead 160 includes an electrically insulative material covering at least some portions of medical lead 160 (e.g., one of the materials listed above). The electrically insulative material covering at least some portions of medical lead 160 can electrically insulate elements disposed within medical lead medical lead 160 (e.g., electrically insulate electrically conductive components, such as conductor wires 180, from blood or other tissue, such as when medical lead 160 is positioned in or advanced through a blood vessel of patient 12).

In the example of FIG. 3A and FIG. 3B, system 100 includes conductor wires 180 (shown individually as conductor wire 180A, conductor wire 180B, conductor wire 180C, conductor wire 180D, conductor wire 180E, conductor wire 180F, conductor wire 180G, and conductor wire 180H, but collectively referred to as conductor wires 180) configured to electrically connect electrodes 170 to a medical device (e.g., medical device 14 of FIG. 1). Each of conductor wires 180A-180H can extend along (e.g., within) at least a portion of medical lead 160. In some examples, some or all of conductor wires 180 are part of medical lead 160, while in other examples, some or all of conductor wires 180 are separate components from medical lead 160.

In some examples, at least a portion of each of conductor wires 180A-180H are housed by the insulative material of medical 160. For example, each of conductor wires 180A-180H can extend within a lumen of medical lead 160. In some examples, insulative material of medical lead 160 can be configured to electrically insulate portions of conductor wires 180 that run along the length of medical lead 160. As shown in the examples of FIG. 3A and FIG. 3B, each of conductor wires 180A-180H can extend distally of medical lead 160, such as to branch out to mechanically connect and/or electrically connect to one or more of electrodes 170. In some examples, each of conductor wires 180A-180H extends along at least a portion of expandable structure 190.

In some examples, some or all of conductor wires 180 include a material or combination of materials configured to facilitate relatively high flexibility, high axial extensibility, and/or high fatigue resistance. For example, one or more wires of conductor wires 180 includes a beta-titanium alloy. In some examples, the beta-titanium alloy comprises a Ti-15Mo alloy. Certain beta-titanium alloys, including Ti-15Mo alloy and similar titanium alloys enable higher wire count coils (e.g., twelve wire or greater, including equal to or greater than sixteen wire coils), such as for situations in which a relatively high number of individually controlled electrodes are needed in a small space including nerve stimulation and/or sensing from endovascular locations. In some examples, one or more of conductor wires 180 includes a core material (e.g., a core at a radial center of each wire). The core material can be configured to enhance mechanical robustness. In some examples, the core material includes tantalum.

In some examples, each of conductor wires 180A-180H can individually and/or collectively be configured to maintain mechanical robustness (e.g., avoid fatigue), even during navigation of endovascular therapy system 100 through a vascular of a patient (e.g., patient 12), deployment of expandable structure 190, and/or long-term or short-term implantation in the presence of blood in vasculature of patient 12. For example, in some examples, at least a portion of conductor wires 180 form a multi-wire coil 182. By forming multi-wire coil 182, individual conductor wires 180 of multi-wire coil 182 may be relatively less prone to mechanical fatigue during bending, axial extension, axial compression, and/or other forces applied to conductor wires 180 (e.g., as compared to examples in which conductor wires 180 do not form multi-wire could 182). In some examples, multi-wire coil 182 extends along at least a portion of medical lead 160 (e.g., the elongated body of medical lead 160, as shown in FIG. 3A and FIG. 3B). In some examples, multi-wire coil 182 extends distally of elongated body distal end 164 of medical lead 160. In some examples, multi-wire coil 182 extends distally of an expandable structure proximal end 193 of expandable structure 190 (e.g., such that a portion of multi-wire coil 182 extends into and resides within an expandable structure lumen 195 defined by expandable structure 190).

As shown in the example of FIG. 3A and FIG. 3B, at least a subset of conductor wires 180 branch out from multi-wire coil 182 to respective electrodes of electrodes 170 (e.g., to electrically connect to the respective electrodes of electrodes 170). By maintaining the coil form-factor of multi-wire coil 182 to a relatively distal location (e.g., at least distally of expandable structure proximal end 193), conductor wires 180 can be less prone to fatigue as compared to other systems in which conductor wires 180 branch out to connect to individual electrodes 170 proximal to or at the junction of medical lead 160 and expandable structure 190.

In some examples, system 100 includes one or more structural elements (e.g., conductor wire management tools) configured to facilitate spacing, organization, and/or routing of conductor wires 180. For example, in some examples, system 100 includes at least one conductor wire separator 166 configured to facilitate splitting (e.g., branching) of individual conductor wires 180A-180H from multi-wire coil 182. In the example shown in FIG. 3B, conductor wire separator 166 is positioned at and/or proximate to a coil transition portion 184. In some examples, coil transition portion 184 defines a transition between multi-wire coil 182 and branched-out portions conductor wires 180A-180H. In the example of FIG. 3A and FIG. 3B, one conductor wire separator 166 is positioned proximate elongated body distal end 164 of medical lead 160 (e.g., proximate the location where each of individual conductor wires 180A-180H exits medical lead 160 and branches out to electrically connect to respective electrodes 170A-170H). In some examples, conductor wire separator 166 is positioned distally of the mechanical connection point between medical lead 160 and expandable structure 190.

In some examples, at least a portion of conductor wire separator 166 is positioned within expandable structure lumen 195 defined by expandable structure 190. For example, conductor wire separator 166 can entirely reside within expandable structure lumen 195. As another example, only part of conductor wire separator 166 can reside within expandable structure lumen 195.

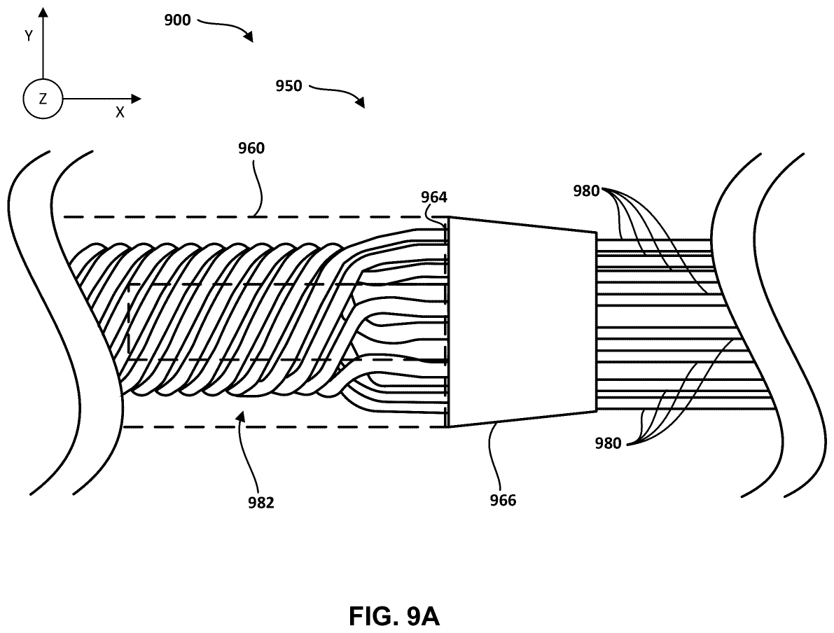

In some examples, conductor wires 180 include one or more coatings. In some examples, a coating applied to conductor wires 180 includes one or more of an antithrombotic (also referred to as antithrombogenic) coating (e.g., to prevent or eliminate the incidence of thrombosis), an electrically insulative coating, a slip coat (e.g., hydrophilic coating), or a suitable combination thereof. As described in connection with FIG. 9A, FIG. 9B, and FIG. 9C, conductor wire separator 166 can be configured to facilitate application of the one or more coatings to conductor wires 180. For example, conductor wire separator 166 can be configured to hold conductor wires 180 physically spaced apart such that one or more coatings can be applied to an exterior surface of each of conductor wires 180.

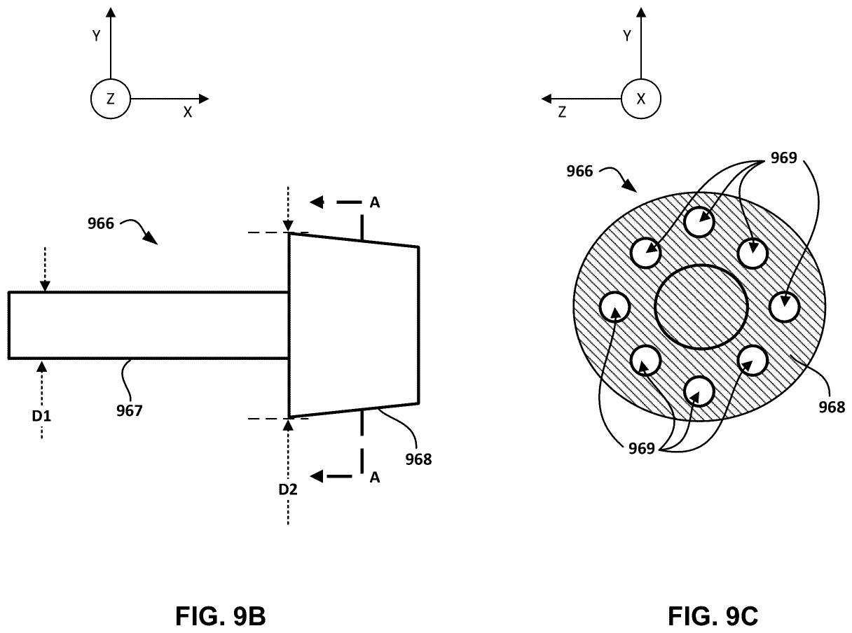

As described in more detail with respect to FIG. 9A, FIG. 9B, FIG. 9C, FIG. 10A, and FIG. 10B, conductor wire separator 166 can be configured to reduce or even eliminate mechanical load placed on conductor wires 180 due to crossing and/or tangling of conductor wires 180A-180H (e.g., such as at coil transition portion 184). In some examples, conductor wire separator 166 can reduce or eliminate a likelihood of unwanted shorting between two or more of conductor wires 180A-180H (e.g., due to degradation of electrically insulative material surrounding individual conductor wires 180A-180H that can cause unwanted electrical contact between individual conductor wires 180A-180H).

In some examples, conductor wires 180 are configured to accommodate the transformation of the expandable structure 190 between the relatively low-profile delivery configuration to the deployed configuration. For example, at least a portion of each of conductor wires 180 extending along expandable structure 190 can include sufficient slack (e.g., sufficient length) to accommodate transformation of the expandable structure 190 between the relatively low-profile delivery configuration to the deployed configuration (e.g., such that each of conductor wires 180A-180H are subjected to little or no tensile force when expandable structure 190 is the in the deployed configuration. The slack can be provided by the length of each of the conductor wires 180 between conductor wire separator 166 and the respective electrode 170A-170H, by configuring the individual conductor wire to effectively lengthen (e.g., due to a shape, such as an undulating shape or coiled shape, or elasticity of the conductor wire), or the like or combination thereof.

In some examples, at least a portion of conductor wires 180A-180H can be affixed to, but able to slide relative to, expandable structure 190. For example, one or more of conductor wires 180A-180H can be affixed to one or more struts 192 of expandable structure 190 that allows the respective conductor wires 180A-180H to slide relative to struts 192 of expandable structure 190 (e.g., when expandable structure 190 transforms between the relatively low-profile delivery configuration to the deployed configuration). In some examples, at least a portion of each of conductor wires 180A-180H are fixed to and follow along struts 192. In other examples, a portion of one or more of conductor wires 180A-180H is fixedly mechanically coupled to a portion of expandable structure 190 (e.g., one or more struts 192 of expandable structure 190) such that the portion of one or more of conductor wires 180A-180H is not able to move relative to expandable structure 190.

In the example of FIG. 3A and FIG. 3B, individual conductor wires 180A-180H generally define straight (or relatively straight) form factors and/or undefined form factors after branching from multi-wire coil 182. However, in other examples, conductor wires 180A-180H can additionally or alternatively define other shapes and/or include other form factors (e.g., that accommodate transformation of the expandable structure 190 between the relatively low-profile delivery configuration to the deployed configuration, such as by effectively lengthening). For example, at least a portion of conductor wires 180A-180H (e.g., the portion of conductor wires 180A-180H that branch out from and are distal to multi-wire coil 182) can define a sinusoidal shape (e.g., planar sinusoidal shape), corrugated shape, or other undulating shapes, uncoiled, semi-straight, or other suitable shapes and/or form factors.

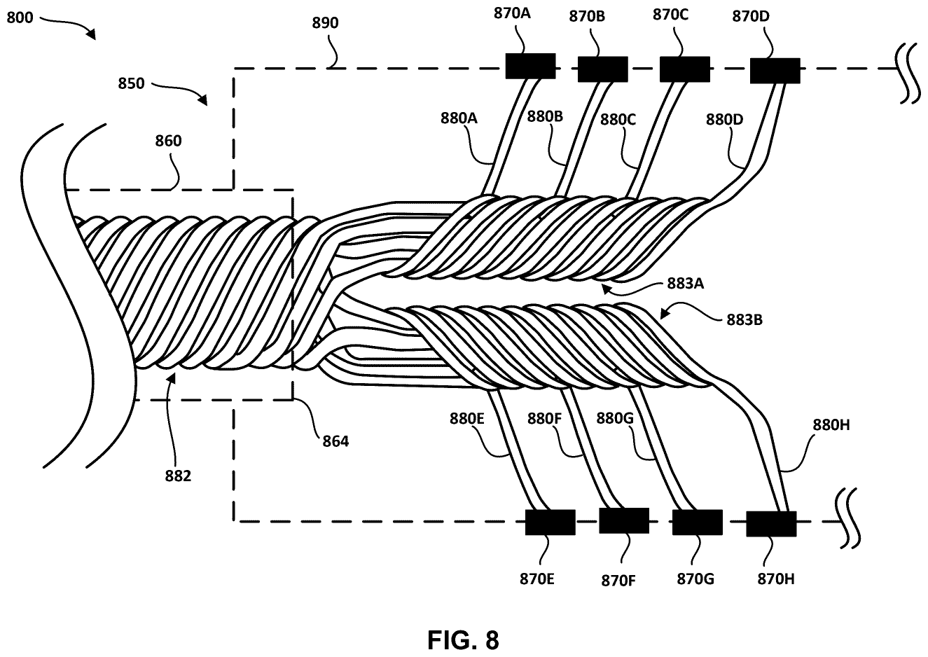

In some examples, as discussed in more detail below, at least some individual conductor wires 180A-180H can form sub-coils or individual wire coils (e.g., after branching out from multi-wire coil 182). For example, as discussed further in relation to FIG. 8, at least a subset of conductor wires 180A-180H can form smaller multi-wire coils (e.g., sub-coils, such as at least a first sub-coil and a second sub-coil) along expandable structure 190 after branching out from multi-wire coil 182 (e.g., such that the sub-coils allow for some extension and compression of some individual conductor wires 180A-180H during transformation of the expandable structure 190 between the relatively low-profile delivery configuration to the deployed configuration). In some examples, at least some individual conductor wires 180A-180H can form a single-wire coil (e.g., such as along a portion expandable structure 190 in a direction parallel to central longitudinal axis 191, after branching out from multi-wire coil 182 that extends along the elongated body of medical lead 160).

In some examples, each of conductor wires 180A-180H are electrically connected to respective electrode electrodes 170A-170H. In some examples, each of electrodes 170A-170H is configured to receive and/or otherwise mechanically couple to one or more conductor wires of conductor wires 180A-180H (e.g., to facilitate the electrical connection between each of conductor wires 180A-180H and one or more of electrodes 170). For example, each electrodes 170A-170H can define one or more conductor holes configured to receive one or more conductor wires 180A-180H, such as for electrically coupling respective electrodes to a medical device (e.g., medical device 14 of FIG. 1). The holes in each of electrodes 170 (e.g., that are configured for receiving and/or electrically connecting to one or more conductor wires 180) can extend partially or entirely though each of electrodes 170. Electrodes 170 can include an electrical contact portion configured to facilitate electrical connection to conductor wires 180.

In some examples, more than one of electrodes 170 are electrically connected to a common conductor wire of conductor wires 180 (e.g., some of electrodes 170 can be “shorted” together). For example, one of conductor wires 180A-180H can be configured to connect to a least a first electrode and a second electrode of electrodes 170 (e.g., such that a medical device 14 can simultaneously control each of the first electrode and the second electrode of electrodes 170 together). Shorting of at least some of electrodes 170 can facilitate control of multiple electrodes at the same time (e.g., for delivery of electrical stimulation therapy and/or sensing).

In some examples, electrodes 170 are carried by expandable structure 190, and expandable structure 190 is configured to position and/or orient electrodes 170 within vasculature of a patient (e.g., patient 12 of FIG. 1). In some examples, at least some of electrodes 170 are carried by and/or mechanically connected to struts 192 of expandable structure 190. In some examples, electrodes 170 are carried by and/or disposed on expandable structure 190, and expandable structure 190 is configured to transform from a relatively low-profile delivery configuration to a deployed configuration in a blood vessel of a patient (e.g., within jugular vein 13 of patient 12 as discussed in relation to FIG. 1 and/or within a cranial blood vessel of patient 12).

Expandable structure 190 is an example of expandable structure 19 as discussed in connection with FIG. 1, and can include any suitable shape and materials. Expandable structure 190 can have any suitable configuration for positioning electrodes 170 for delivering stimulation therapy and/or sensing one or more patient parameters of patient 12 from an endovascular location. In some examples, as shown in FIG. 3A, expandable structure 190 includes a body portion extending between expandable structure proximal end 193 and an expandable structure distal end 194. In some examples, as shown in the FIG. 3A and FIG. 3B, expandable structure 190 includes a plurality of interconnected struts 192. In some examples, expandable structure 190 defines a tubular structure defining an expandable structure lumen 195. In some examples, medical lead 160 is mechanically coupled to expandable structure 190 such that at least a portion of medical lead (e.g., elongated body distal end 164 of medical lead 160) is positioned within expandable structure lumen 195. In some examples, struts 192 are interconnected to form the tubular (e.g., stent-like) structure. In some examples, expandable structure 190 defines a central longitudinal axis 191.

In some examples, expandable structure 190 is configured to expand (e.g., self-expand and/or via an expansion mechanism such as a balloon) radially outward from central longitudinal axis 191 to a deployed configuration, such as to position electrodes 170 into apposition with a blood vessel wall (e.g., for delivering electrical stimulation therapy to tissue of patient 12 proximate the blood vessel and/or sensing a patient parameter from a location within the blood vessel). For example, expandable structure 190 can include one or more of a self-expanding structure, including one or more of a self-expanding stent, a self-expanding coil, or another suitable expandable structure that includes one or more struts as described herein.

Expandable structure 190 can each include suitable configurations for mechanically coupling to and/or carrying one or more electrodes of electrodes 170. In some examples, expandable structure 190 includes structural features configured to facilitate mechanical coupling of electrodes 170 to expandable structure 190, as well as orient electrodes 170 with respect to expandable structure 190. In some examples, one or more of struts 192 are configured to mechanically couple one or more electrodes 170. In addition to or instead of struts 192, in some examples, expandable structure 190 includes other structures (e.g., weld pads, projections, or other structural features) configured to receive, mechanically to, or otherwise carry one or more of electrodes 170. Expandable structure 190 can also be configured to orient electrodes 170 to face radially outward from central longitudinal axis 191 (e.g., when expandable structure 190 is in the deployed configuration). In some examples, expandable structure 190 is configured to position electrodes 170 in apposition with a blood vessel wall (e.g., after system 100 including expandable structure 190 is advanced proximate to a target location in the vasculature of a patient, such as patient 12 of FIG. 1).

Endovascular therapy system 100, including expandable structure 190, may be configured to have a relatively low-profile configuration to facilitate delivery and/or placement into relatively narrow and/or tortuous vessels. Once proximate a target location, expandable structure 190 can be configured to transform to the deployed configuration (e.g., to position the one or more of electrodes 170 to deliver electrical stimulation to tissue of patient 12 or sense a patient parameter from a location within the blood vessel). In some examples, when expandable structure 190 is in a deployed configuration, electrodes 170 are flush or nearly flush with an outer surface of expandable structure 190. In some examples, expandable structure 190 is configured to position electrodes 170 such that at least one surface of each of electrodes 170 is flush or nearly flush with the outer surface of expandable structure 190 (e.g., when expandable structure 190 is in the deployed configuration).

Endovascular therapy system 100 can include any suitable number of electrodes 170 for delivery of stimulation therapy (e.g., electrical stimulation therapy) and/or sensing from an endovascular location. While the example of FIG. 3A and FIG. 3B illustrate endovascular therapy system as including eight of electrodes 170, endovascular therapy system 100 can include any suitable number of electrodes 170 (e.g., one electrode, two electrodes, three electrodes, four electrodes, five electrodes, six electrodes, seven electrodes, eight electrodes, nine electrodes, ten electrodes, twelve electrodes, fifteen electrodes, twenty electrodes, thirty electrodes, etc.). Each of electrodes 170 can be disposed at respective spaced-apart locations along and/or around expandable structure 190.

Expandable structure 190 can include electrodes 170 at multiple circumferential positions around expandable structure 190 and/or multiple longitudinal positions along expandable structure 190 (e.g., spaced apart along central longitudinal axis 191). Longitudinal spacing and/or circumferential spacing between electrodes 170 can correspond to desired longitudinal spacing and/or circumferential spacing between electrode 170 for therapeutically effective endovascular stimulation and/or sensing. In some examples, circumferential spacing between adjacent electrodes (e.g., once mechanically coupled to expandable structure 190 and with no other intervening electrodes) is about 4 millimeters (mm) to about 8 mm around expandable structure 190. In some examples, an axial spacing and/or longitudinal spacing between adjacent electrodes 170 is about 5 mm to about 10 mm. In some examples, a circumferential spacing between adjacent electrodes 170 is less than an axial spacing and/or longitudinal spacing between adjacent electrodes 170. Although referred to as circumferential positions, in some examples, expandable structure 190 is not circular in cross-section (the cross-section being taken in a direction orthogonal to central longitudinal axis 191). In such examples, the circumferential positions may still refer to the rotational position about central longitudinal axis 191).

In some examples, as described in relation to later examples, electrodes 170 can form an electrode array. In some examples, each of electrodes 170 in the electrode array generally face outward in a common radial direction (e.g., in a common radial direction outward from central longitudinal axis 191 of expandable structure 190). Expandable structure 190 can be configured to be rotated (e.g., within the vasculature of patient 12 as described with respect to FIG. 1) to position the electrode array (e.g., including electrodes) 170 to face toward a target location and/or anatomical structure (e.g., toward vagus nerve 21 in the example of FIG. 1).

Electrodes 170 can be fabricated using any suitable method. In some examples, electrodes 170 are formed from a suitable machining (milling, turning, grinding, or electrical discharge machining) and/or stamping process. Electrodes 170 can be formed separately from, and subsequently mechanically coupled to, expandable structure 190. In other examples, electrodes 170 are integrally formed with expandable structure 190.

In some examples, one or more elements of therapy system 100 are configured to facilitate positioning of electrodes 170 at the target site (e.g., via radiographic and/or radiopaque portions that indicate a positioning of electrodes 170). For example, therapy system 100 can include a radiographic or radiopaque marker to indicate an axial position and/or circumferential position of one or more of electrodes 170. In some examples, at least a portion of therapy system 100 is aligned with one or more of electrodes 170 (and/or circumferentially aligned an array of electrodes formed from a group of electrodes 170) to indicate a direction (e.g., a radial direction) faced by electrodes 170. In some examples, therapy system 100 (e.g., one or more components of therapy system 100) includes a radiographic marker that is circumferentially aligned with one or more of electrodes 170 and/or an array of electrodes 170. For example, as discussed in relation to later examples, one or more of medical lead 160 and/or expandable structure 190 (or sub-components thereof) includes a radiographic or radiopaque material circumferentially aligned with electrodes 170 and configured to indicate a radial direction (e.g., a radial direction outwards from central longitudinal axis 191) faced by electrodes 170 (e.g., when expandable structure 190 is in the deployed configuration).

According to the techniques of this disclosure, expandable structure 190 is configured to be mechanically coupled (e.g., directly mechanically coupled without any intervening structures) to medical lead 160 to enable endovascular therapy system 100 to be relatively mechanically robust during delivery and deployment as well as reduce the risk of thrombus formation. In examples described herein, expandable structure 190 is mechanically coupled (e.g., directly mechanically coupled) to medical lead 160 via one or more of a welded connection, a crimped connection, a bonded connection (e.g., via the use of adhesive), and/or a suitable combination or sub-combination thereof. In some examples, a mechanical connection between medical lead 160 and expandable structure 190 facilitates transfer of mechanical forces and/or loads between medical lead 160 and expandable structure 190 while simultaneously reducing a corresponding transfer of mechanical forces and/or loads to conductor wires 180 (which extend along and between each of medical lead 160 and expandable structure 190). In this way, a mechanical connection between medical lead 160 and expandable structure 190 can facilitate a reduction or elimination of mechanical forces and/or loads on conductor wires 180 (e.g., as compared to other medical device systems in which medical lead 160 and expandable structure 190 are not directly mechanically connected).

In some examples, expandable structure 190 is mechanically coupled to medical lead 160 (e.g., the elongated body portion of medical lead 160) such that elongated body distal end 164 is distal to expandable structure proximal end 193. In such a configuration in which elongated body distal end 164 is distal to expandable structure proximal end 193, system 100 can exhibit a relatively greater mechanical integrity as compared to other configurations (e.g., as compared to other examples in which medical lead 160 is mechanically connected to expandable structure 190 such that elongated body distal end 164 is proximal to expandable structure proximal end 193 and/or abutting expandable structure proximal end 193). By keeping elongated body distal end 164 distal to expandable structure proximal end 193, a gap between elongated body distal end 164 and expandable structure proximal end 193 or reduced or eliminated, which can reduce mechanical load (e.g., strain) on conductor wires 180 extending from medical lead 160 to electrodes 170. Such configurations in which the gap between elongated body distal end 164 and expandable structure proximal end 193 is reduced or eliminated can reduce or eliminate an axial mechanical load placed on one or more conductor wires 180 by either of medical lead 160 and/or expandable structure 190, such as when medical lead 160 and/or expandable structure 190 are advanced through vasculature of patient 12.

As another example, by keeping elongated body distal end 164 distal to expandable structure proximal end 193, multi-wire coil 182 can extend further distally into expandable structure 190 before each of conductor wires 180A-180H branch from multi-wire coil 182 to electrically connect to respective electrodes 170A-170H. In this way, the mechanical coupling between medical lead 160 and the expandable structure 190 can reduce a mechanical load on conductor wires 180 and/or reduce fatigue of conductor wires 180, even during navigation of medical lead 160 with conductor wires 180 through vasculature of patient 12 (e.g., because multi-wire coil 182 extends relatively more distal as compared to other medical device system). Similarly, the mechanical coupling between medical lead 160 and the expandable structure 190 can reduce a mechanical load on conductor wires 180 and/or reduce fatigue of conductor wires 180 during transformation of expandable structure 190 between the delivery configuration and the deployed configuration. Similarly, the mechanical coupling between medical lead 160 and expandable structure 190 can reduce a mechanical load on conductor wires 180 and/or reduce fatigue of conductor wires 180 while medical lead 160 and the expandable structure 190 are positioned in patient 12, such as mechanical loading and/or fatigue due to forces from blood of patient 12 moving through vasculature, other movement of patient 12, and/or another source of mechanical loading to or fatigue of conductor wires 180.

In some examples, reduced mechanical load and/or fatigue of conductor wires 180 can enable the individual conductor wires 180 to be relatively thinner (e.g., have a reduced cross-sectional area), which may reduce the overall profile (e.g., cross-sectional profile) of system 100. A reduced overall profile of system 100 can facilitate relatively easier deliverability (e.g., deliverability within vasculature of patient 12) and/or deployment of system 100 proximate a target location, as well as reduce the risk of thrombus formation after system 100 has been delivered and deployed at the target location. Further, the use of relatively thinner conductor wires 180 can enable the use of a greater number of conductor wires 180 (e.g., a relatively greater number of conductor wires 180, as compared to other medical device system), which may in turn enable the use of a greater number of electrodes 170 and/or sensing elements. The use of more electrodes 170 and/or sensing elements can facilitate a greater therapeutic effect of electrical stimulation therapy, more granular measurements from sensed signals, or improvement of other clinically relevant outcomes.

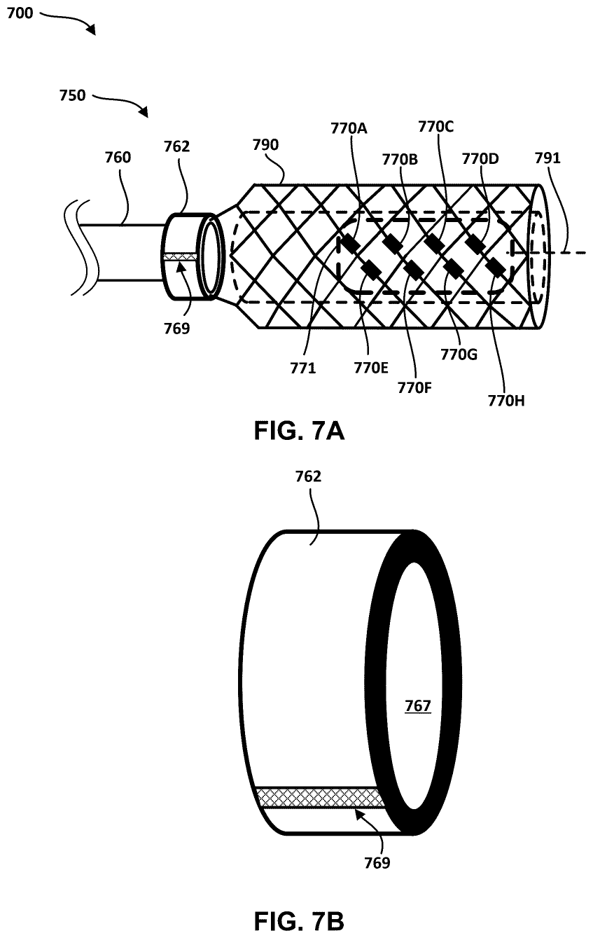

In some examples, medical lead 160 includes a connection element 162 configured to facilitate a mechanical coupling (e.g., via a mechanical connection) between medical lead 160 and expandable structure 190. In some examples, as discussed more fully below, connection element 162 includes one or more of a ring (e.g., a metallic ring), a markerband, a laser-cut tube, or the like. In some examples, at least a portion of connection element 162 is radiopaque (e.g., such as is discussed with respect to the example of FIG. 7A and FIG. 7B). In some examples, as described more fully below, connection element 162 is configured to mechanically couple to one or more portions of expandable structure 190.

In some examples, connection element 162 is fixedly coupled and/or integrally formed with a portion medical lead 160 (e.g., fixedly coupled and/or integrally formed with the elongated body of medical lead 160). In some examples, connection element 162 is positioned at or proximate to distal portion 150 of medical lead 160. In some examples, connection element 162 is concentric with medical lead (e.g., such as in cases in which connection element 162 includes a circular cross section or nearly circular cross section). In some examples, such as the example of FIG. 3A, connection element 162 is positioned proximally of elongated body distal end 164 (e.g., such that a distalmost end of elongated body distal end is distal to a distalmost end of connection element 162).

In some examples, such as the example of FIG. 3A, conductor wire separator 166 is positioned distally of connection element 162. In other examples, such as is described with respect to FIG. 10A and FIG. 10B, conductor wire separator 166 and connection element 162 can be formed as an assembly and/or be positioned at approximately similar axial (e.g., longitudinal) locations relative to medical lead 160 (e.g., at the same axial location along elongated body central longitudinal axis 161 of medical lead 160).

In some examples, connection element 162 is configured to facilitate one or more of a welded connection, a crimped connection, or another suitable mechanical connection between medical lead 160 and expandable structure 190. In some examples, connection element 162 is configured to mechanically couple to expandable structure proximal end 193 of expandable structure 190. In some examples, expandable structure 190 includes a plurality of end portions 196 (shown individually as end portion 196A, end portion 196B, end portion 196C, but collectively referred to herein as end portions 196), and a portion of medical lead 160 (e.g., connection element 162) is configured to mechanically couple to one or more (e.g., at least some or all) of end portions 196 of expandable structure 190. For example, connection element 162 can be configured to mechanically couple to (e.g., directly mechanically couple to) one or more of end portions 196 of expandable structure 190 via a welded connection, a crimped connection, a bonded connection (e.g., via an adhesive and/or another suitable bonding agent), or another suitable mechanical connection.

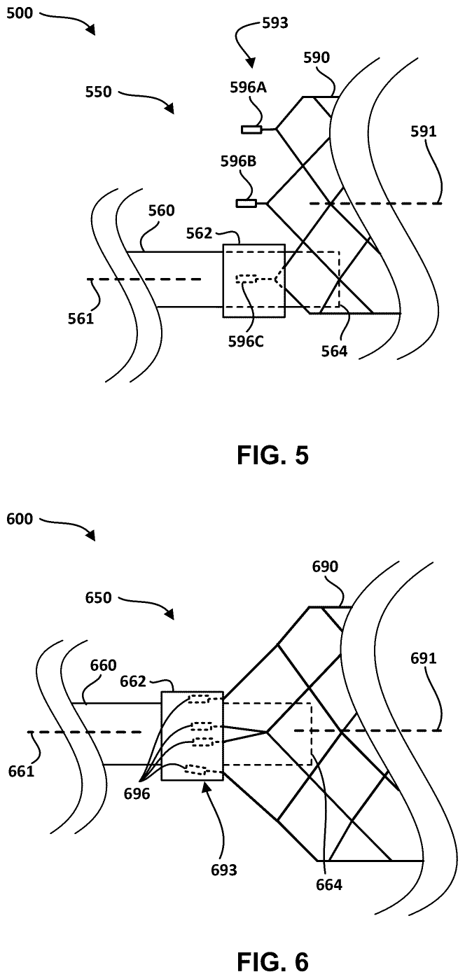

As shown in the example of FIG. 3A, connection element 162 is mechanically coupled to less than all of end portions 196 (i.e., connection element 162 is mechanically coupled to only end portion 196B). However, in other examples (e.g., as discussed with respect to the example of FIG. 6), in which an expandable structure includes multiple end portions 196, connection element 162 can be configured to mechanically couple to more than one of end portions 196 (e.g., which can include all of end portions 196).

End portions 196 of expandable structure 190 can be configured to facilitate mechanical coupling between expandable structure 190 and medical lead 160. In some examples, end portions 196 include a suitable material (e.g., metallic material) configured to be welded (e.g., welded to connection element 162 of medical lead 160). In some examples, end portions 196 include nitinol, and/or or another suitable biocompatible material. In some examples, end portions 196 are laser cut, and can be integrally formed with expandable structure 190. In other examples, end portions 196 are physically separate from, but mechanically connected to, expandable structure 190.

End portions 196 can define a suitable shape and/or thickness to facilitate mechanical coupling with medical lead 160. In some examples, each of end portions 196 defines a circular and/or oval-shape (e.g., with a sufficient surface area for a sufficient mechanical bond between one or more of end portions 196 and a portion of medical lead 160, such as connection element 162).

End portions 196 can additionally or alternatively be configured to facilitate (e.g., enable) positioning of expandable structure 190 during a medical procedure. For example, one or more end portions 196 can be formed from and/or include a radiopaque material. End portions 196 (e.g., when formed from or otherwise include a radiopaque material) can indicate a location of expandable structure proximal end 193 (e.g., which may enable a clinician to determine whether expandable structure 190 has been positioned entirely distally of a guide catheter).

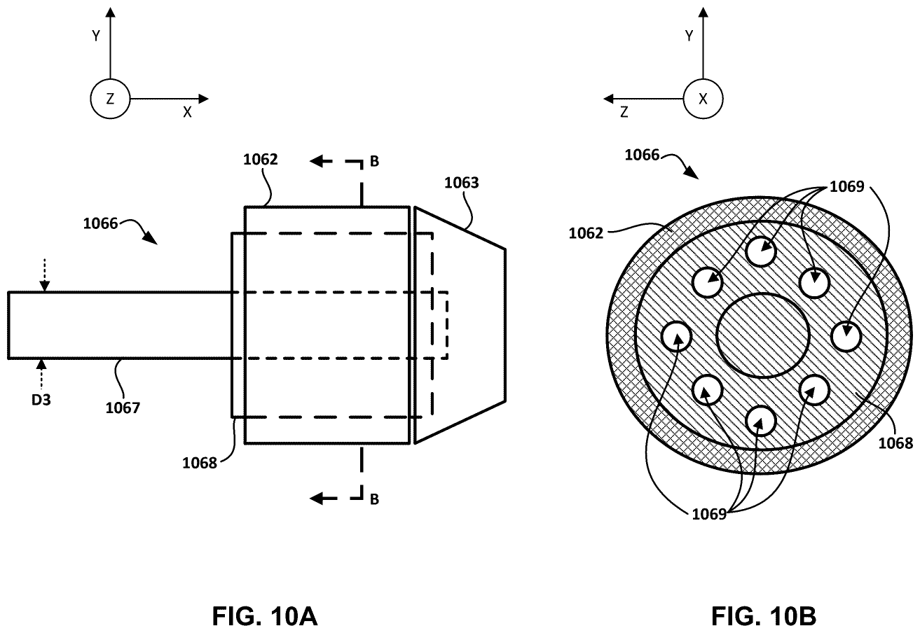

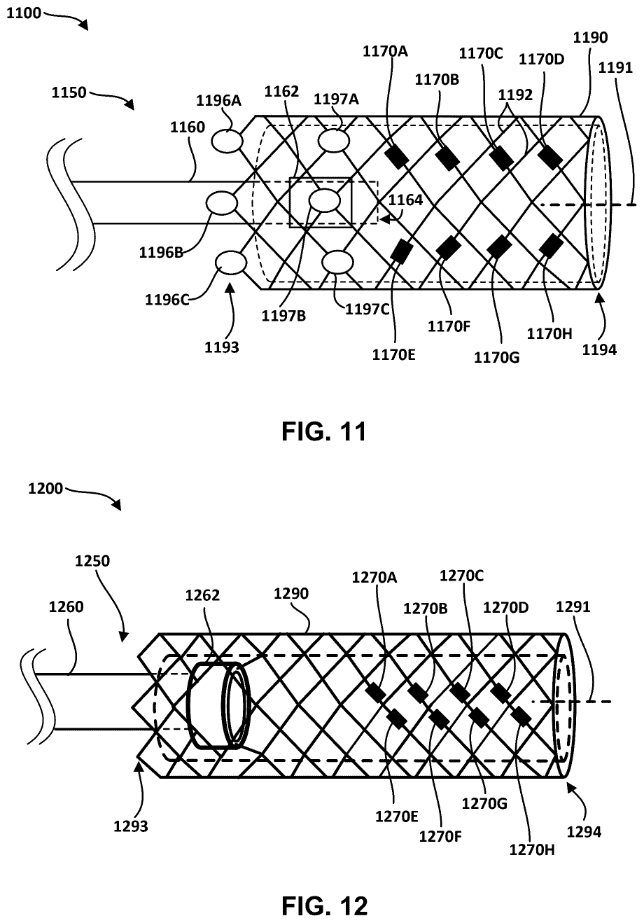

While the example of FIG. 3A illustrates end portions 196 of expandable structure 190 mechanically coupled to medical lead 160, other arrangements are possible. In some examples, other portions of expandable structure 190 (e.g., other than end portions 196) are additionally or alternatively mechanically coupled (e.g., directly mechanically coupled) to a portion of medical lead 160, such as mechanically coupled to connection element 162 of medical lead 160. In some examples, a more distal portion of expandable structure 190 (e.g., more distal portion of expandable structure 190 as compared to end portions 196) is directly mechanically coupled to medical lead 160. For example, as discussed with respect to FIG. 11 and FIG. 12, expandable structure 190 can include one or more additional portions configured to be mechanically coupled with medical lead 160. In some examples, one or more of struts 192 of expandable structure 190 is directly mechanically coupled to medical lead 160. In some examples, medical lead 160 is directly mechanically coupled to expandable structure 190 such that elongated body distal end 164 (e.g., a distalmost end of medical lead 160) is positioned distal to a proximal-most electrode of electrodes 170 (e.g., electrode 170A or electrode 170E of FIG. 3A). In some examples, medical lead 160 is directly mechanically coupled to expandable structure 190 such that elongated body distal end 164 (e.g., a distalmost end of medical lead 160) is positioned distal to a distal-most electrode of electrodes 170 (e.g., electrode 170D or electrode 170H of FIG. 3A). In some examples, medical lead 160 is directly mechanically coupled to expandable structure 190 such that elongated body distal end 164 (e.g., a distalmost end of medical lead 160) is positioned distal to a proximal-most electrode of electrodes 170 (e.g., electrode 170A or electrode 170E of FIG. 3A) but proximal to a distal-most electrode of electrodes 170 (e.g., electrode 170D or electrode 170H of FIG. 3A).

Although FIG. 3A and FIG. 3B is described with respect to electrodes 170 that are configured to deliver electrical stimulation therapy and/or sense electrical signals, endovascular therapy system 100 can additionally or alternatively include other types of therapy delivery elements and/or sensors. In some examples, endovascular therapy system 100 includes one or more ultrasound transducers, chemical delivery elements (e.g., fluid delivery elements and/or drug elution elements) which can be configured to be attached to expandable structure 190 using a similar method of attachment as electrodes 170. In some examples, endovascular therapy system 100 additionally or alternatively includes one or more temperature sensors, pressure sensors, optical sensors, impedance sensors, chemical sensors, and/or other suitable types of sensors, which can be configured to be attached to expandable structure 190 using a similar method of attachment as electrodes 170.

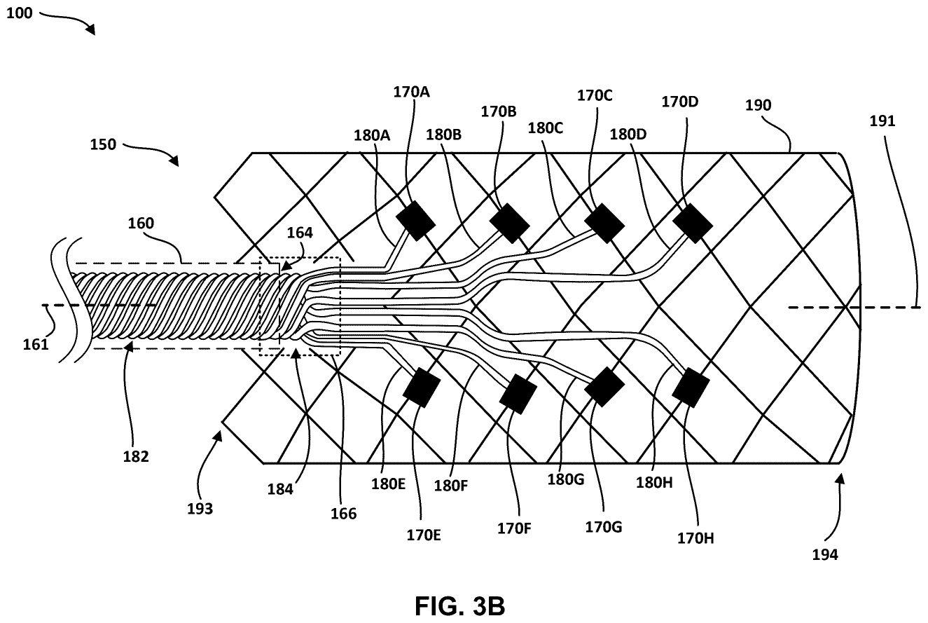

FIG. 4 illustrates an example endovascular therapy system 400, which is an example of therapy system 10 of FIG. 1 and/or therapy system 100 of FIG. 3A and FIG. 3B. Therapy system 400 includes a medical lead 460 and an expandable structure 490 at a distal portion 450 of medical lead 460. Medical lead 460, expandable structure 490, and distal portion 450 are examples of endovascular device 16, expandable structure 19, and distal portion 15 as shown and described in connection with FIG. 1, respectively, except as described herein. Additionally or alternatively, medical lead 460, expandable structure 490, and distal portion 450 are examples of medical lead 160, expandable structure 190, and distal portion 150 as shown and described in connection with FIG. 3A and FIG. 3B, respectively, except as described herein. In the example of FIG. 4, medical lead 460 includes connection element 462, which may be an example of connection element 162 from the example of FIG. 3A and FIG. 3B.

As shown in the example of FIG. 4, expandable structure 490 is mechanically coupled to a portion of medical lead 460 (e.g., connection element 462) such that an elongated body distal end 464 (e.g., a distalmost end of medical lead 460) is distal to an expandable structure proximal end 493 (e.g., a proximalmost end of expandable structure 490).

In some examples, expandable structure 490 includes a plurality of end portions 496 (shown individually as end portion 496A, end portion 496B, end portion 496C, but collectively referred to herein as end portions 496). End portions 496 of expandable structure 490 can be examples of end portions 196 discussed with respect to FIG. 3A and FIG. 3B, except as discussed herein. In some examples, at least some (e.g., less than all or all) of end portions 496 are mechanically coupled to medical lead 460 (e.g., mechanically coupled to connection element 462 of medical lead 460, such as by a suitable crimped connection and/or a welded connection). As shown in the example of FIG. 4, a subset (less than all) of end portions 496 are mechanically connected to medical lead 460. For example, as shown in the example of FIG. 4, a single end portion 496B is mechanically coupled to a portion of medical lead 460 (e.g., coupled to a connection element 462).

In the example of FIG. 4, connection element 462 is configured to both facilitate mechanical connection of medical lead 460 to expandable structure 490 as well as facilitate enhanced mechanical integrity for system 400. In some examples, connection element 462 includes a laser-cut tube. In some examples, the laser-cut tube comprises a suitable shape-memory material (e.g., nitinol). In some examples, connection element 462 (e.g., including the laser-cut tube) is configured as a strain relief (e.g., such that connection element 462 can provide strain relief to one or more conductor wires extending within medical lead 460). In some examples, connection element 462 (e.g., including the laser-cut tube) defines a plurality of cuts (e.g., slits or slots), which may enable connection element 462 to reversible deform (e.g., flex). In some examples, connection element 462 (e.g., including the laser-cut tube) is configured to exhibit a greater column strength as compared to other portions of medical lead 460 (e.g., at least another portion of medical lead 460 proximal to connection element 462).

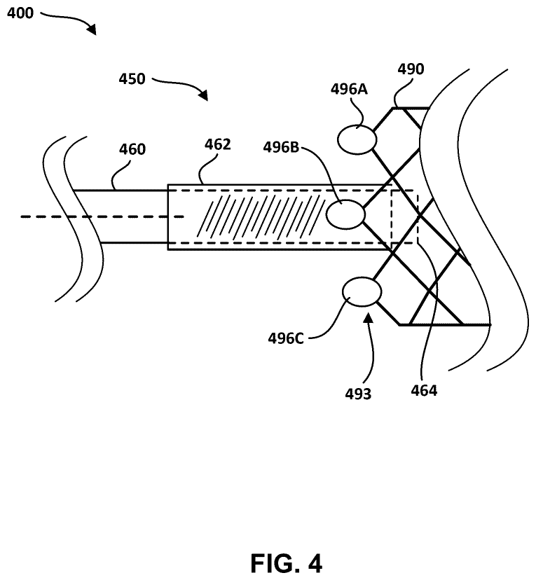

FIG. 5 illustrates an example endovascular therapy system 500, which is an example of therapy system 10 of FIG. 1 and/or therapy system 100 of FIG. 3A and FIG. 3B. FIG. 5 illustrates therapy system 500, which includes a medical lead 560 and an expandable structure 590 at a distal portion 550 of medical lead 560. Medical system 500 can include one or more electrodes disposed on expandable structure 590 (not shown in the example of FIG. 5). Medical lead 560, expandable structure 590, and distal portion 550 are examples of endovascular device 16, expandable structure 19, and distal portion 15 as shown and described in connection with FIG. 1, respectively, except as described herein. Additionally or alternatively, medical lead 560, expandable structure 590, and distal portion 550 are examples of medical lead 160, expandable structure 190, and distal portion 150 as shown and described in connection with FIG. 3A and FIG. 3B, respectively, except as described herein. In the example of FIG. 5, medical lead 560 defines an elongated body central axis 561 extending along the elongated body of medical lead 560 and expandable structure 590 defines a central longitudinal axis 591 (e.g., extending along expandable structure 590 in a direction parallel to elongated body central axis 561).

In the example of FIG. 5, medical lead 560 and expandable structure 590 are mechanically coupled such that medical lead 560 and expandable structure 590 are not coaxial (e.g., axially offset and/or not concentric). That is, in some examples, medical lead 560 and expandable structure 590 are mechanically coupled such that elongated body central axis 561 of medical lead 560 and central longitudinal axis 591 of expandable structure are not coaxial (e.g., are axially offset). Such a configuration can enable medical lead 560 to reside in a portion of a blood vessel (e.g., jugular vein 13) near a wall of the blood vessel (e.g., such that a portion of medical lead 560 abuts the wall of the blood vessel when expandable structure 590 is in a deployed configuration), rather than more centrally in the blood vessel.

For example, when expandable structure 590 is in the deployed configuration (e.g., within a blood vessel such as jugular vein 13), medical lead 560 is mechanically coupled to expandable structure 590 such that at least distal portion 550 of medical lead 560 resides close to and/or abuts the blood vessel wall. In this way, the mechanical connection between the medical lead 560 and expandable structure 590 can enable some portions and/or components of the therapy system 500 to reside closer to a vessel wall and/or apposed to a vessel wall (e.g., a blood vessel wall), which may reduce the impact to blood flow proximate and/or through system 500. In this way, the mechanical coupling between medical lead 560 and expandable structure 590 can facilitate a mechanically robust connection between medical lead 560 and expandable structure 590 while also being relatively low-profile and/or otherwise reducing disruption to blood flow proximate and/or through the expandable structure 590 and/or medical lead 560.

In some examples, expandable structure 590 includes a plurality of end portions 596 (shown individually as end portion 596A, end portion 596B, and end portion 596C, but collectively referred to herein as end portions 596). End portions 596 of expandable structure 590 can be examples of end portions 196 discussed with respect to FIG. 3A and FIG. 3B, except as discussed herein. In some examples, end portions 596 are positioned proximate an expandable structure proximal end 593 of expandable structure 590 (e.g., which may be a proximal-most end of expandable structure 590). In some examples, at least some of end portions 596 are mechanically coupled to medical lead 560 (e.g., mechanically coupled to connection element 562 of medical lead 560, such as by a suitable crimped connection and/or a welded connection). As shown in the example of FIG. 5, a subset (less than all) of end portions 596 are mechanically connected to medical lead 560. For example, a single end portion 596C is mechanically coupled to a portion of medical lead 560 (e.g., mechanically coupled to a connection element 562). Such mechanical coupling of a subset (less than all) of end portions 596 can enable medical lead 560 and expandable structure 590 to be axially offset and/or not concentric, which may reduce impact to blood through and/or proximate to portions of system 500 as discussed above.

In some examples, in which at least some end portions 596 are radiopaque, having less than all end portions 596 mechanically coupled to medical lead 560 can facilitate viewing of expandable structure 590 under a suitable medical imaging modality (e.g., radiography, fluoroscopy, or the like). For example, in which at least some end portions 596 are radiopaque, the subset of end portions 596 not mechanically coupled to medical lead 560 can indicate expansion and axial location of expandable structure 590 and/or radial expansion of at least expandable structure proximal end 593 (e.g., a proximal-most end of expandable structure 590).

In the example of FIG. 5, medical lead 560 and expandable structure 590 are mechanically coupled such that an elongated body distal end 564 of medical lead 560 (e.g., a distalmost end of medical lead 560) is distal to expandable structure proximal end 593 of expandable structure 590. As discussed above with respect to FIG. 3A and FIG. 3B, such a mechanical connection can facilitate a relatively greater mechanical integrity of system 500, e.g., by reducing or eliminating a gap between elongated body distal end 564 and expandable structure proximal end 593.

FIG. 6 illustrates an example endovascular therapy system 600, which is an example of therapy system 10 of FIG. 1 and/or therapy system 100 of FIG. 3A and FIG. 3B. FIG. 6 illustrates therapy system 600, which includes a medical lead 660 and an expandable structure 690 at a distal portion 650 of medical lead 660. System 600 can include one or more electrodes disposed on expandable structure 690 (not shown in FIG. 6). Medical lead 660, expandable structure 690, and distal portion 650 are examples of endovascular device 16, expandable structure 19, and distal portion 15 as shown and described in connection with FIG. 1, respectively, except as described herein. Additionally or alternatively, medical lead 660, expandable structure 690, and distal portion 650 are examples of medical lead 160, expandable structure 190, and distal portion 150 as shown and described in connection with FIG. 3A and FIG. 3B, respectively, except as described herein.

In the example of FIG. 6, medical lead 660 defines an elongated body central axis 661 extending along the elongated body of medical lead 660 and expandable structure 690 defines a central longitudinal axis 691 (e.g., extending along expandable structure 690 in a direction parallel to elongated body central axis 661). In the example of FIG. 6, medical lead 660 and expandable structure 690 are mechanically coupled such that medical lead 660 and expandable structure 690 are axially aligned (e.g., concentric, coaxial and/or not axially offset). That is, in some examples, medical lead 660 and expandable structure 690 are mechanically coupled such that elongated body central axis 661 of medical lead 660 and central longitudinal axis 691 of expandable structure are coaxial (e.g., are not axially offset).

In some examples, expandable structure 690 includes a plurality of end portions 696. End portions 696 of expandable structure 690 can be examples of end portions 196 discussed with respect to FIG. 3A and FIG. 3B, except as discussed herein. In some examples, end portions 696 are positioned proximate an expandable structure proximal end 693 of expandable structure 690 (e.g., which may be a proximal-most end of expandable structure 690). In some examples, at least some of end portions 696 (e.g., some or all) are mechanically coupled to medical lead 660 (e.g., mechanically coupled to connection element 662 of medical lead 660, such as by a suitable crimped connection and/or a welded connection). As shown in the example of FIG. 6, all of end portions 696 are mechanically connected to medical lead 660. Such mechanical coupling of all of end portions 696 can enable medical lead 560 and expandable structure 590 to be axially aligned (e.g., coaxial and/concentric).

In the example of FIG. 6, medical lead 660 and expandable structure 690 are mechanically coupled such that an elongated body distal end 664 of medical lead 660 (e.g., a distalmost end of medical lead 660) is distal to expandable structure proximal end 693 of expandable structure 690. As discussed above with respect to FIG. 3A and FIG. 3B, such a mechanical connection can facilitate a relatively greater mechanical integrity of system 600, e.g., by reducing or eliminating a gap between elongated body distal end 664 and expandable structure proximal end 693.

FIG. 7A illustrates an example endovascular therapy system 700, which is an example of therapy system 10 of FIG. 1 and/or therapy system 100 of FIG. 3A and FIG. 3B. FIG. 7B illustrates a side view of an example connection element 762 from the example of FIG. 7A. FIG. 7A includes a partially schematic diagram of therapy system 700, which includes a medical lead 760, and an expandable structure 790 at a distal portion 750 of medical lead 760. As shown, endovascular therapy system 700 includes electrode 770A, electrode 770B, electrode 770C, electrode 770D, electrode 770E, electrode 770F, electrode 770G, and electrode 770H, collectively referred to herein as electrodes 770. Medical lead 760, expandable structure 790, distal portion 750, and electrodes 770 are examples of endovascular device 16, expandable structure 19, distal portion 15, and electrodes 17 as shown and described in connection with FIG. 1, respectively. Additionally or alternatively, medical lead 760, expandable structure 790, distal portion 750, and electrodes 770 are examples of medical lead 160, expandable structure 190, distal portion 150, and electrodes 170 as shown and described in connection with FIG. 3A and FIG. 3B, respectively.

In the example of FIG. 7A, expandable structure 790 defines a central longitudinal axis 791 and is mechanically connected to distal portion 750 of medical lead 760 via connection element 762. In some examples, expandable structure 790 is mechanically connected to medical lead 760 (e.g., connected to connection element 762 of medical lead 760) via one or more of a welded connection, crimped connection, and/or another suitable bonded connection.

In some examples, connection element 762 includes at least one radiopaque portion 769. In some examples, radiopaque portion 769 includes a strip, a dot, or another suitable shape at least on an outer surface of connection element 762. In some examples, when connection element 762 is mechanically coupled to expandable structure 790, radiopaque portion 769 is circumferentially aligned with one or more of electrodes 770. In some examples, radiopaque portion 769 of connection element 762 is configured as an orientation marker and configured to indicate a circumferential orientation of one or more of electrodes 770 (e.g., a circumferential orientation of electrodes 770 around expandable structure 790 with respect to central longitudinal axis 791). In some examples, radiopaque portion 769 is configured to indicate a radial direction (e.g., a radial direction outward from central longitudinal axis 791 of expandable structure 790) of one or more electrodes. For example, when expandable structure 790 is in the deployed configuration in a blood vessel (e.g., jugular vein 13), radiopaque portion 769 can be circumferentially aligned with one or more of electrodes 770 such that the radial direction faced by one or more of electrodes 770 is parallel to the radial direction faced by radiopaque portion 769 of connection element 762. Such alignment may enable a clinician to rotate expandable structure 790 (e.g., about central longitudinal axis 791) until electrodes 770 face radially outward (e.g., radially outward from central longitudinal axis 791) toward a target location (e.g., toward particular nerves or a brain structure outside of a blood vessel). Such alignment may additionally enable a clinician to confirm that electrodes 770 face radially outward (e.g., radially outward from central longitudinal axis 791) toward the target location (e.g., toward particular nerves outside of a blood vessel) and/or otherwise away from a nontarget location. In this way, radiopaque portion 769 can facilitate directional stimulation or sensing by endovascular therapy system 700.

In some examples, electrodes 770 form an array of electrodes 771, and radiopaque portion 769 of connection element 762 is generally circumferentially aligned with array of electrodes 771. In some examples, at least some of electrodes 770 of array of electrodes 771 are disposed at circumferentially spaced apart locations (e.g., circumferentially spaced apart locations around expandable structure 790), such that the at least some of at least some of electrodes 770 of array of electrodes 771 face radially outward in different directions (e.g., different directions radially outward from central longitudinal axis 791 of expandable structure 790). In some examples in which array of electrodes 771 includes electrodes at different circumferentially spaced apart locations, radiopaque portion 769 of connection element 762 may not be exactly circumferentially aligned with any of electrodes 770, but may still be generally aligned with at least a portion of array of electrodes 771. In some examples, radiopaque portion 769 of connection element 762 is configured as an orientation marker and configured to indicate a circumferential orientation of array of electrodes 771. In examples, in which array of electrodes 771 includes electrodes at different circumferentially spaced apart locations, radiopaque portion 769 of connection element 762 may not be exactly circumferentially aligned with any of electrodes 770, but may still be generally indicate a radial direction (e.g., a radial direction facing outward from central longitudinal axis 791 of expandable structure 790) of array of electrodes 771.

FIG. 7B illustrates connection element 762 from the example of FIG. 7A. Connection element 762 is example of any of the other connection elements discussed in this disclosure (e.g., connection element 162 of FIG. 3A and FIG. 3B, connection element 462 of FIG. 4, connection element 562 of FIG. 5, and/or connection element 662 of FIG. 6, except as described herein). In some examples, connection element 762 includes a tubular structure, e.g., that defines ring shape and/or tubular shape. In some examples, connection element 762 includes radiopaque portion 769 on an outer surface of connection element 762. In some examples, radiopaque portion 769 includes a radiopaque material (e.g., a different material that is more radiopaque as compared to at least another portion of connection element 762).

In some examples, connection element 762 defines a cavity 767. In some examples, cavity 767 is configured to receive and/or mechanically couple to at least a portion of medical lead 760 and/or expandable structure 790 (e.g., such as to mechanically couple medical lead 760 to expandable structure 790 or vice versa).

FIG. 8 illustrates an example endovascular therapy system 800, which is an example of therapy system 10 of FIG. 1 and/or therapy system 100 of FIG. 3A and FIG. 3B. FIG. 8 includes a partially schematic diagram of therapy system 800, which includes a medical lead 860, and an expandable structure 890 at a distal portion 850 of medical lead 860. As shown, endovascular therapy system 800 includes electrode 870A, electrode 870B, electrode 870C, electrode 870D, electrode 870E, electrode 870F, electrode 870G, and electrode 870H, collectively referred to herein as electrodes 870. Medical lead 860, expandable structure 890, distal portion 850, and electrodes 870 are examples of endovascular device 16, expandable structure 19, distal portion 15, and electrodes 17 as shown and described in connection with FIG. 1, respectively. Additionally or alternatively, medical lead 860, expandable structure 890, distal portion 850, and electrodes 870 are examples of medical lead 160, expandable structure 190, distal portion 150, and electrodes 170 as shown and described in connection with FIG. 3A and FIG. 3B, respectively. In the example of FIG. 8, some or all portions of therapy system 800, including medical lead 860 and expandable structure 890, are shown as dashed line representations to facilitate viewing of other structures, however the structure and function of medical lead 860 and/or expandable structure 890 is configured according to the corresponding structures and function of the corresponding structures in FIG. 1 and/or FIG. 3A and FIG. 3B, except as describe herein.

As shown in the example of FIG. 8, system 800 includes a plurality of conductor wires 880 (shown individually as conductor wire 880A, conductor wire 880B, conductor wire 880C, conductor wire 880D, conductor wire 880E, conductor wire 880F, conductor wire 880G, conductor wire 880H, but collectively referred to as conductor wires 880) configured to electrically connect electrodes 870 to a medical device (e.g., medical device 14 in the example of FIG. 1). Each of conductor wires 880A-880H can extend along (e.g., within) at least a portion of medical lead 860. In some examples, each of conductor wires 880A-880H extends along at least a portion of expandable structure 890. As shown in the example of FIG. 8, each of conductor wires 880A-880H can extend distally of the elongated body portion of medical lead 860, such as to branch out to mechanically and/or electrically connect to one or more of electrodes 870.

In some examples, each of conductor wires 880A-880H can individually and/or collectively be configured to maintain mechanical robustness (e.g., avoid fatigue), even during navigation of endovascular therapy system 800 through a vascular of a patient (e.g., patient 12) and/or deployment of expandable structure 890. In some examples, at least a portion of conductor wires 880 form a multi-wire coil 882. By forming multi-wire coil 882, individual conductor wires 880 may be less prone to mechanical fatigue during bending, axial extension, axial compression, or other application of forces to conductor wires 880. In some examples, multi-wire coil 882 extends along at least a portion of the elongated body of medical lead 860.

As shown in the example of FIG. 8, at least some individual conductor wires 180A-180H form at least a first sub-coil 883A and a second sub-coil 883B. In some examples, multi-wire coil 882 branches (e.g., splits) to form first sub-coil 883A and second sub-coil 883B. In some examples, first sub-coil 883A and second sub-coil 883B are positioned distal to multi-wire coil 882 and/or distal to an elongated body distal end 864 (e.g., a distalmost end of medical lead 860). In some examples, one or more of first sub-coil 883A and second sub-coil 883B are not coaxial with multi-wire coil 882. In some examples, each of first sub-coil 883A and second sub-coil 883B allow for some extension and compression of some individual conductor wires 880A-880H during transformation of the expandable structure 890 between the relatively low-profile delivery configuration to the deployed configuration. In some examples, first sub-coil 883A and second sub-coil 883B facilitate reduced mechanical fatigue of individual conductor wires 880A-880H during transformation of the expandable structure 890 between the relatively low-profile delivery configuration to the deployed configuration (e.g., as compared to individual conductor wires 880A-880H in a straight or non-coiled configuration). In some examples, first sub-coil 883A and second sub-coil 883B facilitate reduced mechanical fatigue of individual conductor wires 880A-880H while system 800 is implanted in a patient (e.g., patient 12 of FIG. 1), as each of first sub-coil 883A and second sub-coil 883B can accommodate movement of medical lead 860 and/or expandable structure 890 (e.g., movement such as flexing or other deformation due to movement of the blood vessel in which system 800 is positioned, movement of patient 12, and/or due to other forces and/or mechanical loads placed on medical lead 860 and/or expandable structure 890).

In some examples, each of first sub-coil 883A and second sub-coil 883B include fewer of conductor wires 880 as compared to multi-wire coil 882. In some examples, at least a first subgroup of conductor wires 880 (e.g., in which the first subgroup includes conductor wire 880A, conductor wire 880B, conductor wire 880C, and conductor wire 880D) forms first sub-coil 883A, and a second subgroup of conductor wires 880 (e.g., in which the second subgroup includes conductor wire 880E, conductor wire 880F, conductor wire 880G, and conductor wire 880H) forms second sub-coil 883B.

While the example of FIG. 8 illustrates a single main multi-wire coil 882 branching into two sub-coils (e.g., first sub-coil 883A and second sub-coil 883B) other configurations are contemplated. In some examples, therapy system 800 includes more than one of multi-wire coil 882 (e.g., multiple of multi-wire coil 882 that extend along at least a portion of medical lead 860). In some examples, therapy system 800 includes a different number of sub-coils. In some examples, therapy system 800 includes one sub-coil (e.g., either of first sub-coil 883A and second sub-coil 883B). In some examples, therapy system 800 includes more than two sub-coils (e.g., three, four, five, six, seven, eight, nine, ten, eleven, twelve, thirteen, fourteen fifteen, sixteen, or another suitable number of sub-coils).

Each of multi-wire coil 882, as well as first sub-coil 883A and second sub-coil 883B, can have any suitable number of conductor wires. As shown in the example of FIG. 8, each of first sub-coil 883A and second sub-coil 883B include four of conductor wires 880. However, any number of conductor wires 880 can be used to form sub-coils (e.g., one conductor wire, two conductor wires, three conductor wires, five conductor wires, six conductor wires, or another suitable number of conductor wires). In some examples, each of first sub-coil 883A and second sub-coil 883B include the same number of conductor wires. In other examples, each of first sub-coil 883A and second sub-coil 883B include a different number of conductor wires.

As shown in the example of FIG. 8, individual conductor wires 880 branch from each first sub-coil 883A and second sub-coil 883B to electrically connect to respective electrodes 870. In some examples, each of first sub-coil 883A and second sub-coil 883B define spaced apart exits for individual conductor wires 880 to exit from each of first sub-coil 883A and second sub-coil 883B to electrically connect to respective electrodes 870. As shown in the example of FIG. 8, each of conductor wire 880A, conductor wire 880B, conductor wire 880C, and conductor wire 880D branch from (e.g., exit from) first sub-coil 883A at spaced apart exit points (e.g., spaced apart along expandable structure 890) to electrically connect to respective electrode 870A, electrode 870B, electrode 870C, and electrode 870D. Similarly, each of conductor wire 880E, conductor wire 880F, conductor wire 880G, and conductor wire 880H branch from (e.g., exit from) second sub-coil 883B at spaced apart exit points (e.g., spaced apart along expandable structure 890) to electrically connect to respective electrode 870E, electrode 870F, electrode 870G, and electrode 870H.

FIG. 9A illustrates an example endovascular therapy system 900, which is an example of therapy system 10 of FIG. 1 and/or therapy system 100 of FIG. 3A and FIG. 3B. FIG. 9A includes a side view of a portion of therapy system 900, which includes a medical lead 960, a plurality of conductor wires 980 extending through at least a portion of medical lead 960, and a conductor wire separator 966 at a distal portion 950 of medical lead 960. As shown in FIG. 9A, at least a portion of conductor wires 980 form a multi-wire coil 982. Conductor wires 980 can mechanically and/or electrically connect to respective electrodes (not shown in the example of FIG. 9A). Medical lead 960 and distal portion 950 are examples of endovascular device 16 and distal portion 15, as shown and described in connection with FIG. 1, respectively. Additionally or alternatively, medical lead 960, distal portion 950, conductor wires 980, conductor wire separator 966, and multi-wire coil 982 are examples of medical lead 160, distal portion 150, conductor wires 180, conductor wire separator 166, and multi-wire coil 182 as shown and described in connection with FIG. 3A and FIG. 3B, respectively. In the example of FIG. 9A, some or all portions of therapy system 900, including medical lead 960, are shown as dashed line representations to facilitate viewing of other structures, however the structure and function of medical lead 960 is configured according to the corresponding structures and function of the corresponding structures in FIG. 1 and/or FIG. 3A and FIG. 3B, except as describe herein.

FIG. 9B illustrates a side view of conductor wire separator 966 from the example of FIG. 9A, and FIG. 9C illustrates a section view of conductor wire separator 966 taken through the A-A section lines from the example of FIG. 9B, the section taken in plane parallel to a plane defined by y-axis and z-axis and facing in the negative x-direction according to the orthogonal x-y-z axes in the example of FIG. 9B. In the example of FIG. 9B and FIG. 9C, conductor wire separator 966 includes a proximal body portion 967 and a distal body portion 968.

Conductor wire separator 966 is configured to facilitate splitting (e.g., branching) of multi-wire coil 982, e.g., into individual conductor wires 980 and/or groups of individual conductor wires 980. In the example of FIG. 9A, conductor wire separator 966 is positioned proximate an elongated body distal end 964 of medical lead 960 (e.g., a location where each of individual conductor wires 980 exits medical lead 960). In some examples, a portion of conductor wire separator 966 (e.g., distal body portion 968 of conductor wire separator 966) defines a plurality of conductor holes 969, and each of conductor holes 969 is sized, shaped, and otherwise configured to receive a respective one or more of conductor wires 980 therethrough. In some examples, conductor holes 969 are configured to physically separate (e.g., hold physically spaced apart) at least of a first group of conductor wires 980 from at least a second group of conductor wires 980. For example, conductor holes 969 can be configured to receive respective ones of conductor wires 980 where conductor wires 980 transition from multi-wire coil 982 to other form factors (e.g., sub-coils, individual wire portion, or the like) and keep respective wires or groups of wires of conductor wires 980 physically held separate (e.g., physically spaced apart). In this way, conductor wire separator 966 can be configured to reduce or even eliminate mechanical load placed on conductor wires 180 due to crossing and/or tangling of wires 980 (e.g., such as at coil transition portion 184, as shown and describe with respect to FIG. 3A). In some examples, conductor wire separator 966 can reduce or eliminate a likelihood of unwanted shorting between two or more of conductor wires 980 (e.g., due to degradation of electrically insulative material surrounding individual conductor wires 980 that can cause unwanted electrical connections between individual conductor wires 980).

In some examples, conductor holes 969 can facilitate tracking (e.g., visual distinguishing, such as by a clinician or other user) of individual conductor wires 980 (e.g., such that a user may assess which of conductor wires 980 are mechanically coupled and/or electrically coupled a particular electrode). In some examples, each of conductor holes 969 corresponds to a marking (e.g., a marking on an outer surface of conductor wire separator 966, such as an outer surface of distal body portion 968) that is configured to indicate which of conductor wires 980 is disposed in any given one of conductor holes 969. For example, the marking can include one or more of a number, color, or other indication to track (e.g., visually distinguish) each of conductor wires 980. Such tracking can ensure that corresponding conductor wires 980 are correctly attached to respective electrodes during assembly and/or aid in troubleshooting of conductor wires 980 and/or electrodes.

In some examples, keeping conductor wires 980 physically spaced apart can facilitate application of one or more coatings to conductor wires 980. In some examples, conductor wire separator 966 can facilitate holding conductor wires 980 physically spaced apart such that a coating can be applied (e.g., via a dip coating and/or a spray coating process) around an outer surface (e.g., a circumference) of each of conductor wires 980. For example, when conductor wires 980 are assembled with conductor wire separator 966, a coating can subsequently be applied to at least a portion of each of conductor wires 980 (e.g., such as a portion of conductor wires 980 extending distally of conductor wire separator 966). In some examples, a coating applied to one or more conductor wires 980 includes one or more of an antithrombotic coating (e.g., to prevent or eliminate the incidence of thrombosis), an electrically insulative coating, a slip coat (e.g., hydrophilic coating), or a suitable combination thereof. As compared to when conductor wires 980 are not held physically spaced apart, conductor wire separator 966 can enable application of a coating to a relatively greater surface area (e.g., a surface area of an exterior surface) of conductor wires 980.

In some examples, at least a portion of conductor wire separator 966 is configured to interface with (e.g., fit into, connect with, mechanically couple to, or otherwise physically coordinate with) a portion of medical lead 960. As shown in the examples of FIG. 9A and FIG. 9B, proximal body portion 967 of conductor wire separator 966 can be configured to be inserted into one or more of a lumen defined by medical lead 960 and/or a lumen defined by multi-wire coil 982. In some examples, proximal body portion 967 defines a maximum outer dimension D1 (which may be a diameter in the case of proximal body portion 967 having a circular cross section). In some examples, maximum outer dimension D1 is equal to or smaller than a maximum inner dimension of medical lead 960 and/or a lumen defined by multi-wire coil 982, e.g., such that proximal body portion 967 is configured to be inserted into at least a portion of medical lead 960 and/or a lumen defined by multi-wire coil 982. In some examples, when inserted into multi-wire coil 982, proximal body portion 967 can provide mechanical support (e.g., strain relief) to at least a portion of multi-wire coil 982. Additionally or alternatively, by being configured to be inserted into multi-wire coil 982, proximal body portion 967 can facilitate a relatively easier assembly of system 900 (e.g., because proximal body portion 967 can hold conductor wire separator 966 coaxial with and/or parallel to one or more of multi-wire coil 982 and medical lead 960).

In some examples, distal body portion 968 of conductor wire separator 966 is configured to interface (e.g., fit into, connect with, mechanically couple to, or otherwise physically coordinate with) a portion of medical lead 960. In some examples, distal body portion 968 is configured to abut elongated body distal end 964 of medical lead 960. In some examples, distal body portion 968 defines a defines a maximum outer dimension D2 (which may be a diameter in the case of distal body portion 968 having a circular cross section). In some examples, maximum outer dimension D2 is equal to or greater than a maximum outer dimension of medical lead 960, e.g., such that distal body portion 968 abuts body distal end 964 of medical lead 960 when proximal body portion 967 is inserted into a lumen defined by multi-wire coil 982 and/or a lumen defined by medical lead 960.

In some examples, conductor wire separator 966 is configured to be mechanically coupled to a portion of medical lead 960. In some examples, conductor wire separator 966 is configured to be mechanically coupled to a portion of medical lead 960 via interference fit, a solvent, adhesive bond, reflowed material (e.g., reflowed polymer material to join conductor wire separator 966 to a portion of medical lead 960), another suitable fixation mechanism, or a combination or sub-combination thereof. In some examples, conductor wire separator 966 and medical lead 960 include a common material or common combination of materials (e.g., to facilitate a mechanical robust mechanical connection in the case of adhesive and/or reflowed polymer material used to mechanically connect conductor wire separator 966 and medical lead 960).

FIG. 10A illustrates a side view of another example conductor wire separator 1066, and FIG. 10B illustrates a section view of conductor wire separator 1066 taken through the B-B section lines from the example of FIG. 10A, the section taken in plane parallel to a plane defined by y-axis and z-axis and facing in the negative x-direction according to the orthogonal x-y-z axes in the example of FIG. 10A. Conductor wire separator 1066 is an example of conductor wire separator 166, as shown and described in connection with FIG. 3A and FIG. 3B. Conductor wire separator 1066 can be configured similar to conductor wire separator 966 in the examples of FIG. 9A, FIG. 9B, and FIG. 9C, except as discussed herein. Some portions of FIG. 9A are referenced herein with respect to conductor wire separator 1066, such as to describe wire separator 1066 in connection to other parts of a medical device system, such as system 900 from the example of FIG. 9A. In the example of FIG. 10A and FIG. 10B, conductor wire separator 1066 includes a proximal body portion 1067, a distal body portion 1068, a cover element 1062, and a distal tip portion 1063.

With reference to FIG. 9A, conductor wire separator 1066 is configured to facilitate splitting (e.g., branching) of multi-wire coil 982, e.g., into individual conductor wires 980 and/or groups of individual conductor wires 980. In some examples, conductor wire separator 1066 (e.g., distal body portion 1068) defines a plurality of conductor holes 1069, and each of conductor holes 1069 is sized, shaped, and otherwise configured to receive a respective one or more of conductor wires 980 therethrough. Each of conductor holes 1069 is configured similarly to conductor holes 969 of conductor wire separator 966, as discussed in relation to FIG. 9A, FIG. 9B, and FIG. 9C.

In some examples, at least a portion of conductor wire separator 1066 is configured to interface (e.g., fit into, connect with, mechanically couple to, or otherwise physically coordinate with) a portion of medical lead 960. For example, proximal body portion 1067 can be configured to be inserted into one or more of a lumen defined by medical lead 960 and/or a lumen defined by multi-wire coil 982. In some examples, proximal body portion 1067 defines a maximum outer dimension D3 (which may be a diameter in the case of proximal body portion 1067 having a circular cross section). In some examples, maximum outer dimension D3 is equal to or smaller than a maximum inner dimension of medical lead 960 and/or a lumen defined by multi-wire coil 982, e.g., such that proximal body portion 1067 is configured to be inserted into at least a portion of medical lead 960 and/or a lumen defined by multi-wire coil 982. In some examples, when inserted into multi-wire coil 982, proximal body portion 1067 can provide mechanical support (e.g., strain relief) to at least a portion of multi-wire coil 982. Additionally or alternatively, by being configured to be inserted into multi-wire coil 982, proximal body portion 1067 can facilitate a relatively easier assembly of system 900 (e.g., because proximal body portion 1067 can hold conductor wire separator 1066 coaxial with and/or parallel to one or more of multi-wire coil 982 and medical lead 960).

In some examples, conductor wire separator 1066 is configured to be mechanically coupled to a portion of a medical lead 960. In some examples, conductor wire separator 1066 is configured to be mechanically coupled to a portion of medical lead 960 via interference fit, adhesive bond, reflowed material (e.g., reflowed polymer material to join conductor wire separator 1066 to a portion of medical lead 960), another suitable fixation mechanism, or a combination or sub-combination thereof. In some examples, conductor wire separator 1066 and medical lead 960 include a common material or common combination of materials (e.g., to facilitate a mechanical robust mechanical connection in the case of adhesive and/or reflowed polymer material used to mechanically connect conductor wire separator 1066 and medical lead 960).

In some examples, distal body portion 1068 of conductor wire separator 1066 is configured to interface (e.g., fit into, connect with, mechanically couple to, or otherwise physically coordinate with) a portion of medical lead 960. In some examples, distal body portion 1068 is configured to abut body distal end 964 of medical lead 960. Distal body portion 1068 can be sized, shaped, and/or otherwise configured similarly to distal body portion 968 of conductor wire separator 966 in the example of FIG. 9B and FIG. 9C.

In some examples, distal body portion 1068 of conductor wire separator 1066 is configured to interface (e.g., fit into, connect with, mechanically couple to, or otherwise physically coordinate with) cover element 1062. In some examples, distal body portion 1068 is configured to fit into (e.g., radially inside of) cover element 1062. In some examples, distal body portion 1068 is injected molded into cover element 1062. In some examples, distal body portion 1068 is mechanically coupled to cover element 1062 via press-fit, interference fit, adhesive, another suitable bonding method, or a suitable combination thereof.

In some examples, cover element 1062 is configured to facilitate mechanical coupling of a medical lead to an expandable structure (e.g., medical lead 160 and expandable structure 190 in the example of FIG. 3A and FIG. 3B). For example, with reference to FIG. 3A, cover element 1062 can be configured similarly to connection element 162, except as described herein. With reference to FIG. 3A, in some examples, cover element 1062 is configured to mechanically couple to expandable structure 190, such as to facilitate mechanical coupling of medical lead 160 to expandable structure 190. For example, with reference to FIG. 3A, cover element 1062 can be configured to mechanically couple to at least a portion of expandable structure 190 (e.g., via welding, bonding, adhesive, or another suitable method of mechanical coupling). In some examples, cover element 1062 is configured to mechanically couple to one or more end portions 196 of expandable structure 190. In this way, by serving both to facilitate physical separates of wires (e.g., conductor wires 180 in the example of FIG. 3A and/or conductor wires 980 in the example of FIG. 9A) as well as facilitate mechanical connection between other structures (e.g., mechanical coupling of medical lead 160 to expandable structure 190 from the example of FIG. 3A and FIG. 3B), cover element 1062 can reduce or even eliminate the need for separate structures to serve each of these separate function, which may reduce the overall profile and complexity of a medical device system incorporating cover element 1062.

In some examples, cover element 1062 of conductor wire separator 1066 includes a radiopaque material. In some examples, cover element 1062 is configured as a markerband (e.g., at least a substantial portion or the entirety of cover element 1062 includes a radiopaque material). In some examples, cover element 1062 of conductor wire separator 1066 includes a radiopaque portion including a radiopaque material. For example, cover element 1062 can be configured similarly to connection element 762 including radiopaque portion 769 in the example of FIG. 7A and FIG. 7B (e.g., such that cover element 1062 including a radiopaque portion can be circumferentially aligned with an electrode array).

In some examples, conductor wire separator 1066 includes distal tip portion 1063. In some examples, distal tip portion 1063 is mechanically coupled to distal body portion 1068. In some examples, distal tip portion 1063 positioned distally of distal body portion 1068 and/or distally of cover element 1062. In some examples, distal tip portion 1063 and distal body portion 1068 include a common material or common combination of materials (e.g., to facilitate a mechanical robust mechanical connection in the case of adhesive and/or reflowed polymer material used to mechanically connect distal tip portion 1063 and distal body portion 1068). In some examples, distal tip portion 1063 is molded (e.g., overmolded via injection molding) after cover element 1062 is adjoined to distal body portion 1068. For example, distal tip portion 1063 can be injection molded and mechanically coupled to distal body portion 1068 by placing a sub-assembly of at least distal body portion 1068 and cover element 1062 in a mold, and injecting polymer material into the mold to form distal body portion 1068. In other examples, distal tip portion 1063 is mechanically connected to distal body portion 1068 via one or more of adhesive, bonding (e.g., reflow connection between melted polymer materials), or another suitable mechanical connection.

In some examples, distal tip portion 1063 is configured as a strain relief for one or more elements of system (e.g., strain relief for one or more conductor wires, such as conductor wires 980 as shown in the example of FIG. 9A).

FIG. 11 illustrates an example endovascular therapy system 1100, which is an example of therapy system 10 of FIG. 1. FIG. 11 illustrates a partially schematic diagram of therapy system 1100, which includes a medical lead 1160, and an expandable structure 1190 at a distal portion 1150 of medical lead 1160. As shown, endovascular therapy system 1100 includes electrode 1170A, electrode 1170B, electrode 1170C, electrode 1170D, electrode 1170E, electrode 1170F, electrode 1170G, and electrode 1170H, collectively referred to herein as electrodes 1170. Medical lead 1160, expandable structure 1190, distal portion 1150, and electrodes 1170 are examples of endovascular device 16, expandable structure 19, distal portion 15, and electrodes 17 as shown and described in connection with FIG. 1, respectively.

Medical lead 1160 can have any suitable configuration, and may be configured according to the description of endovascular device 16 of FIG. 1 and/or medical lead 160 of FIG. 3A, except as described herein. In some examples, medical lead 1160 includes an elongated body (e.g., a tubular body defining a lumen) extending between an elongated body proximal end (not show in the examples of FIG. 1) and an elongated body distal end 1164.

As shown in FIG. 11, expandable structure 1190 includes a body portion extending between an expandable structure proximal end 1193 and an expandable structure distal end 1194. Expandable structure 1190 can be configured similarly to any of the expandable structure described in this disclosure, except as described herein.

In the example of FIG. 11, expandable structure 1190 includes a plurality of connected struts 1192 and defines a central longitudinal axis 1191. In some examples, expandable structure 1190 is mechanically connected to distal portion 1150 of medical lead 1160 via connection element 1162. In some examples, expandable structure 1190 is mechanically connected to medical lead 1160 (e.g., connected to connection element 1162 of medical lead 1160) via one or more of a welded connection, crimped connection, and/or another suitable bonded connection.

In some examples, connection element 1162 is configured to facilitate one or more of a welded connection, a crimped connection, or another suitable mechanical connection between medical lead 1160 and expandable structure 1190. Connection element 1162 can be configured similarly to connection element 162 of FIG. 3A, except as described herein. In some examples, connection element 1162 is configured to mechanically couple to an axially intermediate portion of expandable structure 1190 (e.g., a portion of expandable structure 1190 located axially between expandable structure proximal end 1193 and expandable structure distal end 1194). In some examples, expandable structure 190 includes a plurality of connection portions 1197 (shown individually as connection portion 1197A, connection portion 1197B, and connection portion 1197C, but collectively referred to herein as connection portions 1197), and a portion of medical lead 1160 (e.g., connection element 1162) is configured to mechanically couple to one or more (e.g., at least some or all) of connection portions 1197 of expandable structure 1190. For example, connection element 1162 can be configured to mechanically couple to (e.g., directly mechanically couple to) one or more of connection portions 1197 of expandable structure 1190 via a welded connection, a crimped connection, a bonded connection (e.g., via an adhesive and/or another suitable bonding agent), or another suitable mechanical connection.

As shown in the example of FIG. 11, only connection portion is 1197B is mechanically coupled to connection element 1162 of medical lead 1160. In some examples, the other connection portions 1197 not coupled to medical lead 1160 (e.g., connection portion 1197A and connection portion 1197C) can be coupled to additional medical leads like medical lead 1160 and/or can additionally be mechanically coupled to medical lead 1160 (e.g., to serve as a redundant mechanical connection).

In some examples, as shown in the example of FIG. 11, expandable structure 1190 is mechanically connected to medical lead 1160 such that expandable structure proximal end 1193 (which may be a proximal-most end of expandable structure 1190) is proximal to a connection point between medical lead 1160 and expandable structure 1190 (e.g., such that expandable structure proximal end 1193 is entirely proximal to connection element 1162 when expandable structure 1190 is mechanically coupled to medical lead 1160). For example, one or more connection portions 1197 can be positioned on and/or formed by a portion of expandable structure 1190 located axially between expandable structure proximal end 1193 and expandable structure distal end 1194 (e.g., in a direction along central longitudinal axis 1191). Connection portions 1197 can be configured similarly to end portions 196 described with respect to FIG. 3A, such as to facilitate a mechanical connection between medical lead 1160 and expandable structure 1190, except as described herein. For example, connection portions 1197 can define a suitable shape and/or thickness to facilitate mechanical coupling with medical lead 1160 (e.g., each of connection portions 1197 can define a circular and/or oval-shape with a sufficient surface area for a sufficient mechanical bond between at least one of connection portions 1197 and a portion of medical lead 1160, such as connection element 1162). Such configurations can further facilitate a coil of conductor wires (not shown in FIG. 11) housed by medical lead 1160 to extend sufficiently distally before each conductor wire splits out from the coil to electrically connect to respective electrodes 1170.

Connection elements 1197 can have any suitable shape and/or positioning relative to expandable structure 1190. In some examples, connection portion 1197 are positioned at an intersection of two or more of struts 1192. In some examples, connection portion 1197 are posited between adjacent struts of struts 1192. In some examples, at least one of connection portions 1197 is positioned circumferentially between at least some of electrodes 1170.

In some examples, expandable structure 1190 includes a plurality of end portions 1196 (shown individually as end portion 1196A, end portion 1196B, end portion 1196C, but collectively referred to herein as end portions 1196). End portions 1196 can be configured similarly to end portions 196 described with respect to FIG. 3A, except as described herein. In some examples, as shown in the example of FIG. 11, end portions 1196 are not mechanically coupled to medical lead 1160. In other examples, end portions 1196 are configured to be mechanically coupled to a portion of medical lead 1160 (e.g., to connection element 1162 or a separate, more proximally located connection element proximal of connection element 1162), such that medical lead 1160 is mechanically coupled to expandable structure 1190 via at least two connection points (e.g., at least two connection points including at connection portion 1197B and end portion 1196B, which are axially separated by a portion of expandable structure 1190 not mechanically connected to medical lead 1160). Mechanical connection of medical lead 1160 to expandable structure 1190 via at least two axially separated connection points (e.g., including at least a first mechanical connection point and a second mechanical connection point) can serve as a redundant mechanical connection (e.g., which can reduce a likelihood of expandable structure 1190 from being physically separated from medical lead 1160 due to at least one connection point becoming disconnected).

In some examples, end portions 1196 are configured to facilitate (e.g., enable) positioning of expandable structure 1190 during a medical procedure. For example, one or more end portions 1196 can be formed from and/or include a radiopaque material. End portions 1196 (e.g., when formed from or otherwise include a radiopaque material) can indicate a location of expandable structure proximal end 1193 (e.g., which may enable a clinician to determine whether expandable structure 1190 has been positioned entirely distally of a guide catheter).

FIG. 12 illustrates an example endovascular therapy system 1200, which is an example of therapy system 10 of FIG. 1. FIG. 12 illustrates a partially schematic diagram of therapy system 1200, which includes a medical lead 1260, and an expandable structure 1290 at a distal portion 1250 of medical lead 1260. As shown, endovascular therapy system 1200 includes electrode 1270A, electrode 1270B, electrode 1270C, electrode 1270D, electrode 1270E, electrode 1270F, electrode 1270G, and electrode 1270H, collectively referred to herein as electrodes 1270. Medical lead 1260, expandable structure 1290, distal portion 1250, and electrodes 1270 are examples of endovascular device 16, expandable structure 19, distal portion 15, and electrodes 17 as shown and described in connection with FIG. 1, respectively.

As shown in FIG. 12, expandable structure 1290 includes a body portion extending between an expandable structure proximal end 1293 and an expandable structure distal end 1294. Expandable structure 1290 can be configured similarly to any of the expandable structure described in this disclosure, except as described herein.

In the example of FIG. 12, expandable structure 1290 defines a central longitudinal axis 1291 and is mechanically connected to distal portion 1250 of medical lead 1260 via connection element 1262. In some examples, expandable structure 1290 is mechanically connected to medical lead 1260 (e.g., connected to connection element 1262 of medical lead 1260) via one or more of a welded connection, crimped connection, and/or another suitable bonded connection. For example, connection element 1262 can include a ring configured to couple to at least some loose ends (e.g., strut ends) of expandable structure 1290 (not shown in FIG. 12). In some examples, as shown in the example of FIG. 12, expandable structure 1290 is mechanically connected to medical lead 1260 such that expandable structure proximal end 1293 (which may be a proximal-most end of expandable structure 1290) is proximal to a connection point between medical lead 1260 and expandable structure 1290 (e.g., such that expandable structure proximal end 1293 is entirely proximal to connection element 1262 when expandable structure 1290 is mechanically coupled to medical lead 1260). Such configurations can further facilitate a coil of conductor wires (not shown in FIG. 12) housed by medical lead 1260 to extend sufficiently distally before each conductor wire splits out from the coil to electrically connect to respective electrodes 1270.

In some examples, connection element 1262 is configured similar to connection element 762 of FIG. 7A and FIG. 7B and includes one or more radiopaque portions (e.g., like radiopaque portion 769 connection element 762).

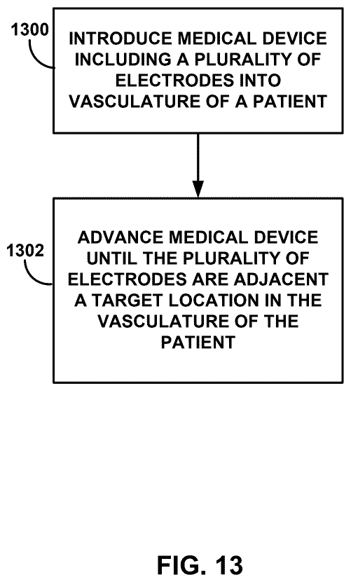

FIG. 13 is a flow diagram illustrating an example technique for using a medical device system according to the techniques of this disclosure, which may include placing a medical lead adjacent a target location in vasculature of a patient. The technique of FIG. 13 is described with respect to therapy system 10 of FIG. 1, as well as endovascular therapy system 100 of FIG. 3A and FIG. 3B (which is an example of therapy system 10 of FIG. 1), but may be used with any of the device, systems, and/or elements of systems described in this disclosure.

In the example of FIG. 13, the technique includes introducing an endovascular device (e.g., endovascular device 16 and/or medical lead 160) into vasculature of patient 12 (1300). For example, a clinician may introduce at least distal portion 150 medical lead 160 through an access point in patient 12 including a femoral artery access point or radial artery access point. In some examples, one or more of an introducer sheath, a guide catheter, and/or a guidewire is used to facilitate introduction of medical lead 160 into patient 12.

In the example of FIG. 13, the technique further includes advancing medical lead 160 through the vasculature of the patient until electrodes 170 are adjacent a target location in the vasculature of patient 12 (1302). In some examples, a clinician advances medical lead 160 through vasculature of patient 12 until electrodes 170 are located within jugular vein 13 and positioned adjacent vagus nerve 21. In other examples, a clinician advances medical lead 160 through vasculature of patient 12 until electrodes 170 are located within a cranial blood vessel proximate one or more target brain structures. Once electrodes 170 are adjacent the target location (e.g., vagus nerve 21, other nerve, or one or more brain structures), the clinician initiates (e.g., via programmer 20, or another suitable device) electrical stimulation therapy and/or sensing of one or more patient parameters by medical device 14 via electrodes 170.

In some examples, expandable structure 190, which can be at a distal portion of medical lead 160, is configured transform from a relatively low-profile delivery configuration to a deployed configuration in a blood vessel of a patient (e.g., within jugular vein 13 of patient 12). In some examples, expandable structure 190 remains in the delivery configuration during advancement of medical lead through the vasculature.

In some examples, a clinician causes expandable structure 190 to transform to the deployed (e.g., expanded) configuration once electrodes 170 are adjacent the target site. In the deployed configuration of expandable structure 190, one or more of electrodes 170 can be positioned into apposition with the vessel wall (e.g., the vessel wall of jugular vein 13). In some examples, electrodes 170 are configured to bias transmissions of electrical signals to and/or sensing of signals from tissue surrounding the blood vessel (jugular vein 13) as compared to radially inward from the blood vessel wall (e.g., towards central longitudinal axis 191). For example, electrodes 170 can be configured with an electrically insulative material applied to a radially inward surface of each of electrodes 170. The biasing of transmissions of electrical signals to and/or from tissue surrounding the blood vessel can help facilitate directional electrical stimulation and/or sensing. In this way, the endovascular devices described herein can help target any suitable target tissue sites (e.g., nerve structures and/or brain structures) from an endovascular location and/or help avoid nontarget tissue sites, e.g., those associated with negative side effects.

In some examples, one or more elements of therapy system 100 is configured to facilitate positioning of electrodes 170 at the target site (e.g., via radiographic and/or radiopaque portions that indicate a positioning of electrodes 170). In some examples, at least a portion of therapy system 100 is aligned with one or more of electrodes 170 (and/or circumferentially aligned an array of electrodes formed from a group of electrodes 170) to indicate a direction (e.g., a radial direction) faced by electrodes 170. In some examples, therapy system 100 (e.g., one or more components of therapy system 100) includes a radiographic or radiopaque marker that is circumferentially aligned with one or more of electrodes 170 and/or an electrode array formed by electrodes 170. In some examples, one or more of medical lead 160, connection element 162, and/or expandable structure 190 includes a radiographic or radiopaque material circumferentially aligned with electrodes 170 and configured to indicate a radial direction (e.g., a radial direction outwards from central longitudinal axis 191) faced by electrodes 170 (e.g., when expandable structure 190 is in the deployed configuration).

The techniques described in this disclosure, including those attributed to medical device 14, programmer 20, or various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as clinician or patient programmers, medical devices, or other devices. Processing circuitry, control circuitry, and sensing circuitry, as well as other processors and controllers described herein, may be implemented at least in part as, or include, one or more executable applications, application modules, libraries, classes, methods, objects, routines, subroutines, firmware, and/or embedded code, for example. In addition, analog circuits, components and circuit elements may be employed to construct one, some or all of the processing circuitry 30, instead of or in addition to the partially or wholly digital hardware and/or software described herein. Accordingly, analog or digital hardware may be employed, or a combination of the two.

In one or more examples, the functions described in this disclosure may be implemented in hardware, software, firmware, or any combination thereof. If implemented in software, the functions may be stored on, as one or more instructions or code, a computer-readable medium and executed by a hardware-based processing unit. The computer-readable medium may be an article of manufacture including a non-transitory computer-readable storage medium encoded with instructions. Instructions embedded or encoded in an article of manufacture including a non-transitory computer-readable storage medium encoded, may cause one or more programmable processors, or other processors, to implement one or more of the techniques described herein, such as when instructions included or encoded in the non-transitory computer-readable storage medium are executed by the one or more processors. Example non-transitory computer-readable storage media may include RAM, ROM, programmable ROM (PROM), erasable programmable ROM (EPROM), electronically erasable programmable ROM (EEPROM), flash memory, a hard disk, a compact disc ROM (CD-ROM), a floppy disk, a cassette, magnetic media, optical media, or any other computer readable storage devices or tangible computer readable media.

In some examples, a computer-readable storage medium comprises non-transitory medium. The term “non-transitory” may indicate that the storage medium is not embodied in a carrier wave or a propagated signal. In certain examples, a non-transitory storage medium stores data that can, over time, change (e.g., in RAM or cache).

The functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements.

This disclosure includes the following non-limiting examples.

Example 1: An endovascular medical device system includes an elongated body configured to be introduced in a blood vessel of a patient; an expandable structure at a distal portion of the elongated body, the expandable structure including a plurality of interconnected struts and extending from an expandable structure proximal end to an expandable structure distal end; a plurality of electrodes carried by the expandable structure; and a plurality of conductor wires, each conductor wire of the plurality of conductor wires electrically connected to respective electrode of the plurality of electrodes, wherein the plurality of conductor wires collectively form a multi-wire coil, the multi-wire coil extending along at least a portion of the elongated body and extending distally of the expandable structure proximal end, and wherein at least a subset of conductor wires of the plurality of conductor wires branch out from the multi-wire coil to the respective electrodes of the plurality of electrodes to electrically connect to the respective electrodes.

Example 2: The endovascular medical device system of example 1, wherein the expandable structure is configured to transform from a relatively low-profile delivery configuration to a deployed configuration to position the plurality of electrodes to deliver electrical stimulation to tissue of the patient or sense a patient parameter from a location within the blood vessel, and wherein the plurality of conductor wires is configured to accommodate the transformation of the expandable structure from the relatively low-profile delivery configuration to the deployed configuration.

Example 3: The endovascular medical device system of any of examples 1 and 2, wherein the elongated body extends between an elongated body proximal end and an elongated body distal end, and wherein the expandable structure is coupled to the elongated body such that the elongated body distal end is distal to the expandable structure proximal end.

Example 4: The endovascular medical device system of any of examples 1 through 3, wherein the elongated body includes a connection element at the distal portion of the elongated body, and wherein the expandable structure is coupled to the connection element of the elongated body via a mechanical connection, the mechanical connection including one or more of a welded connection or a crimped connection.

Example 5: The endovascular medical device system of example 4, wherein the mechanical connection includes the welded connection between the expandable structure and the connection element of the elongated body.

Example 6: The endovascular medical device system of example 4, wherein the mechanical connection includes the crimped connection between the expandable structure and the connection element of the elongated body.

Example 7: The endovascular medical device system of any of examples 4 through 6, wherein the expandable structure includes a plurality of end portions, and wherein at least some of the plurality of end portions are mechanically coupled to the connection element of the elongated body.

Example 8: The endovascular medical device system of example 7, wherein all end portions of the plurality of end portions are mechanically coupled to the connection element of the elongated body.

Example 9: The endovascular medical device system of any of examples 4 through 8, wherein the connection element includes a laser-cut tube, the laser-cut tube configured as a strain relief.

Example 10: The endovascular medical device system of any of examples 4 through 9, wherein the connection element includes a radiopaque portion.

Example 11: The endovascular medical device system of example 10, wherein the radiopaque portion of the connection element is configured as an orientation marker, the orientation marker configured to indicate a circumferential orientation of the plurality of electrodes.

Example 12: The endovascular medical device system of any of examples 2 through 11, wherein when the expandable structure is in the deployed configuration, the elongated body and the expandable structure are coaxial.

Example 13: The endovascular medical device system of any of examples 2 through 11, wherein when the expandable structure is in the deployed configuration, the elongated body and the expandable structure are not coaxial.

Example 14: The endovascular medical device system of any of examples 1 through 13, further comprising a conductor wire separator, the conductor wire separator configured to facilitate splitting of the multi-wire coil into individual conductor wires.

Example 15: The endovascular medical device system of example 14, wherein the conductor wire separator includes a portion configured to be inserted into a lumen defined by the multi-wire coil.

Example 16: The endovascular medical device system of any of examples 14 and 15, wherein the conductor wire separator includes a radiopaque material.

Example 17: The endovascular medical device system of any of examples 1 through 16, wherein the multi-wire coil branches into at least a first sub-coil and a second sub-coil, and wherein individual conductor wires branch from each of the first sub-coil and the second sub-coil to electrically connect to respective electrodes of the plurality of electrodes.

Example 18: A method of using a medical device system includes introducing a medical device into vasculature of a patient, the medical device includes an elongated body configured to be introduced in a blood vessel of a patient; an expandable structure at a distal portion of the elongated body, the expandable structure including a plurality of interconnected struts and extending from an expandable structure proximal end to an expandable structure distal end; a plurality of electrodes carried by the expandable structure; and a plurality of conductor wires, each conductor wire of the plurality of conductor wires electrically connected to respective electrode of the plurality of electrodes, wherein the plurality of conductor wires collectively form a multi-wire coil, the multi-wire coil extending along at least a portion of the elongated body and extending distally of the expandable structure proximal end, and wherein at least a subset of conductor wires of the plurality of conductor wires branch out from the multi-wire coil to the respective electrodes of the plurality of electrodes to electrically connect to the respective electrodes; and advancing the medical device until the plurality of electrodes are at or near a target location in the vasculature of the patient.

Example 19: The method of example 18, wherein the expandable structure is configured to transform from a relatively low-profile delivery configuration to a deployed configuration to position the plurality of electrodes to deliver electrical stimulation to tissue of the patient or sense a patient parameter from a location within the blood vessel, and wherein the plurality of conductor wires is configured to accommodate the transformation of the expandable structure from the relatively low-profile delivery configuration to the deployed configuration.

Example 20: The method of any of examples 18 and 19, wherein the elongated body extends between an elongated body proximal end and an elongated body distal end, and wherein the expandable structure is coupled to the elongated body such that the elongated body distal end is distal to the expandable structure proximal end.

Example 21: The method of any of examples 18 through 20, wherein the elongated body includes a connection element at the distal portion of the elongated body, and wherein the expandable structure is coupled to the connection element of the elongated body via a mechanical connection, the mechanical connection including one or more of a welded connection or a crimped connection.

Example 22: The method of example 21, wherein the mechanical connection includes the welded connection between the expandable structure and the connection element of the elongated body.

Example 23: The method of example 21, wherein the mechanical connection includes the crimped connection between the expandable structure and the connection element of the elongated body.

Example 24: The method of any of examples 21 through 23, wherein the expandable structure includes a plurality of end portions, and wherein at least some of the plurality of end portions are mechanically coupled to the connection element of the elongated body.

Example 25: The method of example 24, wherein all end portions of the plurality of end portions are mechanically coupled to the connection element of the elongated body.

Example 26: The method of any of examples 21 through 25, wherein the connection element includes a laser-cut tube, the laser-cut tube configured as a strain relief.

Example 27: The method of any of examples 21 through 26, wherein the connection element includes a radiopaque portion.

Example 28: The method of example 27, wherein the radiopaque portion of the connection element is configured as an orientation marker, the orientation marker configured to indicate a circumferential orientation of the plurality of electrodes.

Example 29: The method of any of examples 19 through 28, wherein when the expandable structure is in the deployed configuration, the elongated body and the expandable structure are coaxial.

Example 30: The method of any of examples 19 through 28, wherein when the expandable structure is in the deployed configuration, the elongated body and the expandable structure are not coaxial.

Example 31: The method of any of examples 18 through 30, further comprising a conductor wire separator, the conductor wire separator configured to facilitate splitting of the multi-wire coil into individual conductor wires.

Example 32: The method of example 31, wherein the conductor wire separator includes a portion configured to be inserted into a lumen defined by the multi-wire coil.

Example 33: The method of any of examples 31 and 32, wherein the conductor wire separator includes a radiopaque material.

Example 34: The method of any of examples 18 through 33, wherein the multi-wire coil branches into at least a first sub-coil and a second sub-coil, and wherein individual conductor wires branch from each of the first sub-coil and the second sub-coil to electrically connect to respective electrodes of the plurality of electrodes.

Example 35: An endovascular medical device system includes an elongated body configured to be introduced in a blood vessel of a patient, the elongated body extending between an elongated body proximal end and an elongated body distal end; an expandable structure extending from an expandable structure proximal end to an expandable structure distal end, wherein the expandable structure is mechanically connected to the elongated body such that the elongated body distal end is distal to the expandable structure proximal end; a plurality of electrodes carried by the expandable structure; and a plurality of conductor wires, each conductor wire of the plurality of conductor wires electrically connected to respective electrodes of the plurality of electrodes, wherein the plurality of conductor wires collectively form a multi-wire coil, the multi-wire coil extending along at least a portion of the elongated body and extending distally of the expandable structure proximal end, wherein at least a subset of conductor wires of plurality of conductor wires branch out from the multi-wire coil to the respective electrodes of the plurality of electrodes to electrically connect to the respective electrodes, wherein the expandable structure is configured to transform from a relatively low-profile delivery configuration to a deployed configuration to position the plurality of electrodes to deliver electrical stimulation to tissue of the patient or sense a patient parameter from a location within the blood vessel, and wherein the plurality of conductor wires is configured to accommodate the transformation of the expandable structure from the relatively low-profile delivery configuration to the deployed configuration.

Example 36: The endovascular medical device system of example 35, wherein the expandable structure including a plurality of interconnected struts, and wherein at least some electrodes are carried by one or more struts of the interconnected struts.

Various examples of the disclosure have been described. Any combination of the described systems, operations, or functions is contemplated. These and other examples are within the scope of the following claims.

What is claimed is:

1. An endovascular medical device system comprising:
an elongated body configured to be introduced in a blood vessel of a patient;
an expandable structure at a distal portion of the elongated body, the expandable structure including a plurality of interconnected struts and extending from an expandable structure proximal end to an expandable structure distal end;
a plurality of electrodes carried by the expandable structure; and
a plurality of conductor wires, each conductor wire of the plurality of conductor wires electrically connected to respective electrode of the plurality of electrodes,
wherein the plurality of conductor wires collectively form a multi-wire coil, the multi-wire coil extending along at least a portion of the elongated body and extending distally of the expandable structure proximal end, and
wherein at least a subset of conductor wires of the plurality of conductor wires branch out from the multi-wire coil to the respective electrodes of the plurality of electrodes to electrically connect to the respective electrodes.

2. The endovascular medical device system of claim 1,
wherein the expandable structure is configured to transform from a relatively low-profile delivery configuration to a deployed configuration to position the plurality of electrodes to deliver electrical stimulation to tissue of the patient or sense a patient parameter from a location within the blood vessel, and
wherein the plurality of conductor wires is configured to accommodate the transformation of the expandable structure from the relatively low-profile delivery configuration to the deployed configuration.

3. The endovascular medical device system of claim 1, wherein the elongated body extends between an elongated body proximal end and an elongated body distal end, and wherein the expandable structure is coupled to the elongated body such that the elongated body distal end is distal to the expandable structure proximal end.

4. The endovascular medical device system of claim 3,
wherein the elongated body includes a connection element at the distal portion of the elongated body, and
wherein the expandable structure is coupled to the connection element of the elongated body via a mechanical connection, the mechanical connection including one or more of a welded connection or a crimped connection.

5. The endovascular medical device system of claim 4, wherein the mechanical connection includes the welded connection between the expandable structure and the connection element of the elongated body.

6. The endovascular medical device system of claim 4, wherein the mechanical connection includes the crimped connection between the expandable structure and the connection element of the elongated body.

7. The endovascular medical device system of claim 6,
wherein the expandable structure includes a plurality of end portions, and
wherein at least some of the plurality of end portions are mechanically coupled to the connection element of the elongated body.

8. The endovascular medical device system of claim 7, wherein all end portions of the plurality of end portions are mechanically coupled to the connection element of the elongated body.

9. The endovascular medical device system of claim 8, wherein the connection element includes a laser-cut tube, the laser-cut tube configured as a strain relief.

10. The endovascular medical device system of claim 9, wherein the connection element includes a radiopaque portion.

11. The endovascular medical device system of claim 10, wherein the radiopaque portion of the connection element is configured as an orientation marker, the orientation marker configured to indicate a circumferential orientation of the plurality of electrodes.

12. The endovascular medical device system of claim 11, wherein when the expandable structure is in the deployed configuration, the elongated body and the expandable structure are coaxial.

13. The endovascular medical device system of claim 11, wherein when the expandable structure is in the deployed configuration, the elongated body and the expandable structure are not coaxial.

14. The endovascular medical device system of claim 13, further comprising a conductor wire separator, the conductor wire separator configured to facilitate splitting of the multi-wire coil into individual conductor wires.

15. The endovascular medical device system of claim 14, wherein the conductor wire separator includes a portion configured to be inserted into a lumen defined by the multi-wire coil.

16. The endovascular medical device system of claim 15, wherein the conductor wire separator includes a radiopaque material.

17. The endovascular medical device system of claim 16,
wherein the multi-wire coil branches into at least a first sub-coil and a second sub-coil, and
wherein individual conductor wires branch from each of the first sub-coil and the second sub-coil to electrically connect to respective electrodes of the plurality of electrodes.

18. A method of using a medical device system, the method comprising:
introducing a medical device into vasculature of a patient, the medical device comprising:
an elongated body configured to be introduced in a blood vessel of a patient;
an expandable structure at a distal portion of the elongated body, the expandable structure including a plurality of interconnected struts and extending from an expandable structure proximal end to an expandable structure distal end;
a plurality of electrodes carried by the expandable structure; and
a plurality of conductor wires, each conductor wire of the plurality of conductor wires electrically connected to respective electrode of the plurality of electrodes,
wherein the plurality of conductor wires collectively form a multi-wire coil, the multi-wire coil extending along at least a portion of the elongated body and extending distally of the expandable structure proximal end, and
wherein at least a subset of conductor wires of the plurality of conductor wires branch out from the multi-wire coil to the respective electrodes of the plurality of electrodes to electrically connect to the respective electrodes; and
advancing the medical device until the plurality of electrodes are at or near a target location in the vasculature of the patient.

19. The method of claim 18,
wherein the expandable structure is configured to transform from a relatively low-profile delivery configuration to a deployed configuration to position the plurality of electrodes to deliver electrical stimulation to tissue of the patient or sense a patient parameter from a location within the blood vessel, and
wherein the plurality of conductor wires is configured to accommodate the transformation of the expandable structure from the relatively low-profile delivery configuration to the deployed configuration.

20. The method of claim 19, wherein the elongated body extends between an elongated body proximal end and an elongated body distal end, and wherein the expandable structure is coupled to the elongated body such that the elongated body distal end is distal to the expandable structure proximal end.
